(19) United States
(12) Patent Application Publication
(10) Pub. No.: US 2023/0263774 A1
(43) Pub. Date: Aug. 24, 2023

(54) OPTIMIZED SURGERY PROTOCOL AND KITS

(71) Applicant: Steadman Philippon Research Institute, Vail, CO (US)

(72) Inventors: Johnny HUARD, Edwards, CO (US); Marc Joseph PHILIPPON, Edwards, CO (US)

(73) Assignee: Steadman Philippon Research Institute, Vail, CO (US)

(21) Appl. No.: 18/040,959

(22) PCT Filed: Jul. 30, 2021
(86) PCT No.: PCT/US2021/043969

§ 371 Date: Feb. 7, 2023

Related U.S. Application Data
(60) Provisional application No. 63/157,174, filed on Mar. 5, 2021.
(60) Provisional application No. 63/063,271, filed on Aug. 8, 2020.

(51) Int. Cl. A61K 31/4178; A61K 31/353; A61P 19/00; A61P 19/02

(52) U.S. Cl. CPC A61K 31/4178; A61K 31/353; A61P 19/00; A61P 19/02

(57) ABSTRACT

The present disclosure provides methods for enhancing a therapeutic outcome in a subject having a musculoskeletal condition or disorder, comprising administering at least one senolytic agent and/or at least one anti-fibrotic agent to the subject.

This application is being filed on Jul. 30, 2021, as a PCT International Patent Application and claims the benefit of priority to U.S. Provisional Patent Application Nos. 63/063,271, filed on Aug. 8, 2020, and 63/157,174, filed on Mar. 5, 2021; the disclosures of each herein incorporated by reference in their entireties.

The present disclosure was supported in part by National Institutes of Health Grant No. NIH 1UG3AR077748-01, and the government may have certain rights in the present disclosure.

### FIELD OF THE DISCLOSURE

### Background

Osteoarthritis (OA) is a progressive joint disease leading to cartilage damage, pain, and loss of function. There are currently no effective FDA-approved therapies that modify the course of joint destruction from OA. Current surgical approaches to repair damaged/diseased cartilage include bone marrow stimulation, osteochondral allograft transplantation, and autologous chondrocyte implantation. The limitations of these interventions is that they often necessitate total joint replacement. The development of novel strategies, such as adult stem cell transplantation, that could improve articular cartilage (AC) repair after injury and/or prevent the development and progression of osteoarthritis is highly desirable.

Adult stem cells have been identified and harvested from a variety of tissues, including bone marrow, skeletal muscle, adipose tissue, etc. Bone marrow Stem Cells (BMSCs) from bone marrow aspirate concentrate (BMC) offer the significant translational advantage that they can be harvested using minimally invasive technology without the need of in vitro expansion, and they are already being used in the clinic for OA and other applications. There is, however, significant potential for improving efficacy of BMSC treatment for OA. The number of senescent cells in BMC increases with age and OA. In cellular senescence, normally proliferating cells are in a permanent state of cell cycle arrest, in which they no longer respond to growth stimuli and no longer divide. Cell cycle arrest in progenitor cells contributes to a loss in the capacity to repair tissue. Furthermore, senescent cells release pro-inflammatory cytokines/chemokines, proteases, and other senescence-associated secretory phenotypes (SASP) that can impair stem cell function and likely contribute to OA development/progression. Furthermore, transplantation of senescent cells induces an osteoarthritis-like condition in mice (Xu, et al. 2017 J Gerontol A Biol Sci Med Sci 72(6):780-785), demonstrating that SASP factors can contribute to cartilage degeneration (Jeon, et al. 2018 J Clin Invest 128(4):1229-1237).

Transforming growth factor (TGF-β1) is widely believed to be essential for articular cartilage homeostasis. However, it has been shown that inhibition of TGF-β1 signaling protects adult knee joints against the development of osteoarthritis (Chen, et al. 2015 Am J Pathol 185(10:2875-85).

### BRIEF SUMMARY OF THE INVENTION

A high percentage of senescent cells are observed herein in bone marrow stem cells (BMSCs), and elimination of these senescent cells may improve the beneficial effect of BMSCs for osteoarthritis (OA) patients. Compounds have been found that specifically kill senescent cells, abrogating systemic SASP factors (Yousefzadeh, et al. 2018 EBioMedicine 36:18-28; Niedernhofer and Robbins 2018 Nat Rev Drug Discov 17:377). These senolytic agents can delay osteoarthritis (OA) in a preclinical model (NCT04210986; Collins, et al. 2018 Curr Opin Rheumatol 30(1):101-107).

Preclinical work has shown that blocking fibrosis can improve the regenerative potential of adult stem cells in skeletal muscle (Utsunomiya, et al. 2019 Orthopaedic J Sports Med 7(supp15):2325967119S00263). It has also been shown that blocking fibrosis with Losartan, a TGF-β1 blocker, can improve cartilage repair by promoting regeneration of hyaline cartilage while reducing the amount of fibrocartilage. It is disclosed herein that Losartan, a TGF-β1 inhibitor, may also improve the regenerative potential of BMSCs for hyaline cartilage repair and, consequently, improve their benefit for OA patients.

Thus, in one aspect, the disclosure provides a method for enhancing a therapeutic outcome in a subject having a musculoskeletal condition or disorder, comprising administering at least one senolytic agent and/or at least one anti-fibrotic agent to the subject. In specific embodiments of a method according to the invention, the therapeutic outcome is related to the outcome of surgical and/or non-surgical treatment of a bone injury or bone condition or bone disorder. In one embodiment, the non-surgical treatment comprises administration of an orthobiologic to the subject. In another embodiment, the non-surgical treatment comprises administration of bone marrow stem cells to a subject for the treatment of osteoarthritis. In such an embodiment, the method according to the disclosure enhances the beneficial effect of bone marrow stem cells (BMSCs) for treating osteoarthritis (OA) in the subject.

A phase I/II clinical trial has been initiated to evaluate the safety and efficacy of Fisetin, a senolytic dietary supplement, and Losartan, an anti-fibrotic drug, used either individually or in combination, for improving the clinical efficacy of BMSCs in the treatment of knee osteoarthritis (NCT04815902).

Thus, in one embodiment of a method according to the disclosure, the at least one senolytic agent is Fisetin. In another embodiment of a method according to the disclosure, the at least one anti-fibrotic agent is Losartan. In still another embodiment of a method according to the disclosure, the at least one senolytic agent is Fisetin, and the at least one anti-fibrotic agent is Losartan.

In one embodiment of a method according to the disclosure, the senolytic agent is administered to the subject in cycles of about 2 days on/about 28 days off before the treatment/therapy. In another embodiment, the senolytic agent is administered to the subject in cycles of about 2 days on/about 28 days off before and after the treatment/therapy.

In one embodiment of a method according to the disclosure, the anti-fibrotic agent is administered for at least about 30 days after the treatment/therapy. In another embodiment, the anti-fibrotic agent is administered for about 30 days after the treatment/therapy. In still another embodiment, the anti-fibrotic agent is administered every day for the at least about 30 days.

In another embodiment of a method according to the disclosure, the senolytic agent is administered to the subject in cycles of about 2 days on/about 28 days off before the treatment/therapy, and the anti-fibrotic agent is administered for at least about 30 days after the treatment/therapy. In another embodiment, the senolytic agent is administered to the subject in cycles of about 2 days on/about 28 days off before and after the treatment/therapy, and the anti-fibrotic agent is administered for at least about 30 days after the treatment/therapy.

In one embodiment of a method according to the disclosure, the at least one senolytic agent is Fisetin, and it is administered at a dosage of about 1000 mg/day. In another embodiment, the at least one senolytic agent is Fisetin, and it is administered at a dosage of about 10 mg/kg/day to about 100 mg/kg/day. In another embodiment, the at least one senolytic agent is Fisetin, and it is administered at a dosage of about 20 mg/kg/day.

In another embodiment of a method according to the disclosure, the at least one anti-fibrotic agent is Losartan, and it is administered at a dosage of about 10 mg/day to about 200 mg/day. In still another embodiment of a method according to the disclosure, the at least one anti-fibrotic agent is Losartan, and it is administered at a dosage of about 25 mg/day.

In one aspect, the disclosure provides a method for improving the outcome of BMSC treatment of symptomatic knee osteoarthritis in a subject, comprising combining the BMSC treatment with administration of a senolytic agent (for example, Fisetin) to the subject.

In another aspect, the disclosure provides a method for improving the outcome of BMSC treatment of symptomatic knee osteoarthritis in a subject, comprising combining the BMSC treatment with administration of an anti-fibrotic agent (for example, a TGF-β1 inhibitor, for example, Losartan) to the subject.

In still another aspect, the disclosure provides a method for improving the outcome of BMSC treatment of symptomatic knee osteoarthritis in a subject, comprising combining the BMSC treatment with administration of a senolytic agent (for example, Fisetin) and an anti-fibrotic agent (for example, a TGF-β1 inhibitor, for example, Losartan) to the subject. In one embodiment, the combination will result in a synergistic effect comprising the elimination of senescent cells and the reduction of fibrosis, when compared to treatment with BMSC treatment plus either administration of a senolytic agent (for example, Fisetin) or an anti-fibrotic agent (for example, a TGF-β1 inhibitor, for example, Losartan), used individually.

In one aspect, the disclosure provides a method for reducing the senescent cell content and/or SASPs in the peripheral blood mononucleated cells, plasma, and/or serum of a subject having symptomatic knee osteoarthritis, comprising administering a senolytic agent to the subject.

In another aspect, the disclosure provides a method for reducing the senescent cell content and/or SASPs in the bone marrow and/or marrow-derived plasma of a subject having symptomatic knee osteoarthritis, comprising administering a senolytic agent to the subject.

In still another aspect, the disclosure provides a method for reducing the senescent cell content and/or SASPs in the synovial cells and/or fluid of a subject having symptomatic knee osteoarthritis, comprising administering a senolytic agent to the subject.

In one embodiment, a method according to the disclosure further comprises detecting and/or measuring senescent cells in a sample obtained from the subject. In another embodiment, the detecting and/or measuring comprises staining the sample cells with C₁₂FDG; and subjecting the stained cells to flow cytometry. In still another embodiment, the detected senescent cells are characterized according to stage of senescence. In still another embodiment, the characterization is based on brightness of signal. In a further embodiment, the stage of senescence is early-stage (relatively low C₁₂FDG positivity, “dim”, low green fluorescent intensity on a flow cytometry plot), mid-stage (relatively moderate C₁₂FDG positivity), or late-stage (relatively high C₁₂FDG positivity, “bright”, high fluorescent intensity on a flow cytometry plot), as determined by normalized event gating with flow cytometry.

In embodiments of the disclosure that specify the selection of “at least one . . . selected from the group consisting of” or simply “selected from the group consisting of”, the use of the conjunction “and” between the final two items of the list following such language indicates that the items in the sequence are alternatives to one another, and that one (or more) of these items is/are selected. It does not mean that each of the items is necessarily selected.

Other embodiments of the present invention will become apparent from a review of the ensuing detailed description.

### BRIEF DESCRIPTION OF THE FIGURES

FIGS. 1A-1H show that senescent cell transplantation induces osteoarthritis-like phenotypes and impairs function. (FIG. 1A) Safranin 0/Fast Green staining, (FIG. 1B) Histology scores and (FIG. 1C) Representative radiographs are showing senescence induced osteoarthritis-like phenotype. Knee joints are shown as mean±SEM (N=5). Increased paw withdrawal in the von Frey filament frequencies at 0.16 g (FIG. 1D) and 0.4 g (FIG. 1E) with mean±SEM (N=7). (FIG. 1F) Rotarod assay showing percent change in time to falling relative to baseline with mean±SEM (N=7). Locomotor activity evaluation showed Stationary Time (ST) and Active Time (AT) as mean±SEM (N=7) (FIG. 1G). Distance traveled is shown as mean±SEM (N=7) (FIG. 1H).

FIGS. 2A-2D show age-associated differences in SASP factor concentrations. BMA samples were collected from a 47-year-old female and a 20-year-old male <3 months after ACL injury. BMA levels of several SASP factors are show in comparison: (FIG. 2A) MMP-2; (FIG. 2B) MMP-3; (FIG. 2C) RANTES; and (FIG. 2D) MMP-12.

FIGS. 3A and 3B show age-associated differences in senescent CD3⁺ T-cells using C₁₂FDG staining. (FIG. 3A) Detection of C₁₂FDG positive (senescent) cells using flow cytometry. BMA were collected from two different female patients (23-year-old and 47-year-old) with OA. (FIG. 3B) Cells were collected using density gradient centrifugation.

FIG. 4 shows elimination of senescent cells during expansion of ADSCs by senolytic drug treatment to enrich healthy stem cells prior to any clinical application. Senescence is quantified by H2AX and H3K9 staining. Fisetin reduces [symbol] -H2AX and H3K9 in Adipose Derived Stem Cells. (*=p<0.05, **=p<0.1).

FIGS. 5A and 5B show chondrocyte dysfunction and cartilage degeneration in adult Z24−/− mice. (FIG. 5A) pellet culture of isolated chondrocytes were significantly smaller, with decreased Col2 signal and chondrogenic capacity (per toluidine blue stain intensity), (FIG. 5B) Safranin O staining of Z24−/− AC revealed obvious loss of proteoglycan content versus WT at only 5 months of age.

FIG. 6 shows preservation of proteoglycan content in articular cartilage of Z24 mice following single or multi D/Q treatment. Top row shows Alcian Blue staining; bottom row shows Safranin O staining.

FIG. 7 shows decreased expression of OA marker ADMTS4 following D/Q treatment in Z24−/− mice. Left, untreated, right, D/Qₘᵤₗ.

FIGS. 8A-8C show histological outcomes of BRMS by blocking TGF-β1 with Losartan oral administration. FIGS. 8A and 8B show Safranin O staining (FIG. 8A) and O'Driscoll score (FIG. 8B). (*P<0.05, **P<0.01). (FIG. 8C) Immunohistochemistry staining of Col2.

FIGS. 9A-9C show that the histological evaluation of TA muscle showed significantly increased regenerative myofibers in both MDSC and MDSC+Losartan treatments (FIG. 9A). Bigger diameter of new muscle fibers (faster regeneration) was only found in MDSC+Losartan (FIG. 9B) compared to control PBS treatment. Muscles treated with MDSCs+Losartan showed less fibrous scar tissue, compared to MDSCs and PBS treatment (FIG. 9C).*means p<0.05

FIGS. 10A-10C show an assessment of 6 weeks post-operative cartilage repair. (FIG. 10A) macroscopic assessment BMAC(−), left, and BMAC(+), right; (FIG. 10B) microCT BMAC(−), left, and BMAC(+), right; (FIG. 10C) H&E staining BMAC(−), left, and BMAC(+), right.

FIG. 11 shows an assessment of collagen-II in regenerated cartilage.

FIG. 12 shows the antioxidant effect of Fisetin.

FIGS. 13A-13C show chondrocytes showing improved hyaline cartilage morphology. (FIG. 13A) macroscopic assessment BMAC(−), left, and BMAC(+), right; (FIG. 13B) microCT BMAC(−), left, and BMAC(+), right; (FIG. 13C) Alcian blue staining BMAC(−), left, and BMAC(+), right.

FIG. 14 shows T2* mapping relaxation time differences between ACL-reconstructed and contralateral (uninjured) tibial cartilage 24 months after surgery (50 subjects). Significant differences between limbs (starred regions, p<0.05) identified in the central and deep cartilage layers. From left to right, superficial, middle, and deep.

FIG. 15 shows changes in cartilage thickness between 6 and 24 months after ACL reconstruction (average, 50 subjects). Significant cartilage thickening observed for the medial tibial plateau (p<0.05; starred regions).

FIGS. 16A-16E show senescence detection of human peripheral blood cells. (FIG. 16A) Distinct high intensity and lower intensity populations of C₁₂FDG stained PBMCs and T-cells (green, bright; red, dim); (FIG. 16B) % CD3+ T-cells using CD3 specific antibody; (FIG. 16C) Co-expression of C₁₂FDG+/CD26+ in CD3+ T-cells; (FIG. 16D) Co-expression of C₁₂FDG+/CD28+ in CD3+ T-cells showing CD28 loss; and (FIG. 16E) Co-expression of C₁₂FDG+/CD87+ in PBMCs.

FIGS. 17A and 17B show (FIG. 17A) Percent and total count of bright C12FDG cells are associated with Chronological age. Young Age (YA), 20-33; Old Age (OA) 75-87. (FIG. 17B) Heat map demonstrating correlation between C12FDG senescence staining and SASP and aging biomarkers in blood plasma.

FIG. 18 shows the workflow from clinical sample collection (peripheral blood, bone marrow and synovial fluid) for analyses.

FIGS. 19A-19D show PBMC detection with C12FDG using flow cytometry. In FIG. 19A, cells are identified using FSC and SSC controls. (FIG. 19B) PBMCs displayed a distribution of two distinct populations of C₁₂FDG signal. (FIG. 19C) Peaks to show the same. (FIG. 19D) Highly senescent cells were found to correlate with increasing age of study participants.

FIGS. 20A and 20B show FACS sorted highly senescent populations expression profiles. (FIG. 20A) Low, moderate, and high populations were sorted using FACS for two study participants. (FIG. 20B) Expression levels for senescence/SASP markers p16INK4A and IL-1β.

FIGS. 21A-21D show Fisetin effects on highly senescent cells. (FIG. 21A) Fisetin treatment significantly reduced high senescent cell counts and percent senescent cells in as little as 1 hr (middle panel), with a maximum reduction at 4 hrs (righthand panel). (FIG. 21B) Results of 7A in cell counts. (FIG. 21C) Results in % senescent cells. (FIG. 21D) Rate of senolytic activity of Fisetin versus other known senotherapeutic drugs such as metformin, dasatinib, quercetin.

FIGS. 22A-22D show that Fisetin selectively kills highly senescent cells in isolated PBMCs. (FIG. 22A) Decreases in highly senescent cells (high C₁₂FDG intensity) due to Fisetin treatment were associated with concomitant increases in DRAQ7+ cells—decrease in C₁₂FDG intensity at 1 (middle panel) and further decrease at 4 hrs (righthand panel) concomitant with increase in DRAQ7+ cells at 1 and further increase at 4 hrs. (FIG. 22B) Results of 8A in cell counts. (FIG. 22C) Results in % senescent cells. (FIG. 22D) Fisetin effect on highly senescent cells vs moderately senescent cells.

FIGS. 23A and 23B show detection of senescent T-Cells and PBMCs with C₁₂FDG. (FIG. 23A) Flow cytometry analysis results: cells (PBMCs in left panel, T-cells in right panel) were identified using FSC controls, and senescent cells, or C₁₂FDG+ events, were identified with an emission of 514 nm (green channel). (FIG. 23B) Same results quantified.

FIG. 24 shows a correlation of highly senescent T-Cells with plasma biomarkers.

FIGS. 25A and 25B show that Fisetin reduces senescent T-Cells and biomarkers associated with aging and OP. (FIG. 25A) Cell counts over time (of Fisetin dosing). (FIG. 25B) OP markers OPG, OPN, SOST, and TNF-α pre- vs. post-Fisetin.

FIG. 26 shows the collection timepoints for specimen isolation.

FIG. 27 shows reduction in senescent bone marrow mesenchymal stem cells (BM-MSCs) after senolytic treatment. # of senescent cells is shown for DMSO-treated BM-MSCs vs. those treated with CM+FGF vs. those treated with Fisetin+CM+FGF. Upper boxes show dot plots, and lower boxes show histogram plots.

FIG. 28 shows detection of senescent cells in synovial fluid. Both PBMCs (left panel) and synovial fluid (middle and right panels) display two distinct populations of C₁₂FDG signal cells.

FIGS. 29A-29C show detection of senescent cells in joint fluid. (FIG. 31A) Subject 88 within 48 hrs of injury. (FIG. 31B) Subject 20 within 48 hrs of injury. (FIG. 31C) Subject 20 at 6 wks from injury.

FIG. 30 shows SASP associated biomarkers within synovial fluid samples from acute knee injured patients between 20-50 years of age.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present disclosure, the preferred methods and materials are now described. All publications mentioned herein are incorporated herein by reference to describe in their entirety.

### Senescence

Senescence is a state of permanent cell cycle arrest. Senescent cells are incapable of proliferation, but they retain cellular function, metabolic activity, and viability. Cellular senescence drives age-related decline and is associated with many age-associated conditions and disorders, including conditions and disorders of the musculoskeletal system.

Senescent cell burden has been shown to strongly correlate with age-related orthopaedic conditions. The injection of senescent cells is sufficient to drive age-related conditions such as osteoarthritis, frailty, and decreased survival. Thus, the development of therapies that selectively kill senescent cells is anticipated to delay the onset of aging phenotypes, attenuate severity of age-related diseases, improve resiliency, enhance survival, and extend lifespan (Xu, et al. 2018 Nat Med 24:1246-1256; Xu, et al. 2017 J Gerentol A Biol Sci Med Sci 72(6):780-785).

Senescent cells and senescent cell-associated molecules can be detected by techniques and procedures described in the art. For example, the presence of senescent cells in tissues can be analyzed by histochemistry or immunohistochemistry techniques that detect the senescence marker, SA-β galactosidase (SA-β gal) (Dimri, et al. 1995 Proc. Natl Acad. Sci. USA 92:9363-9367; Lee, et al. 2006 Aging Cell 5(2):187-195). The presence of the senescent cell-associated polypeptide p16, specifically, p16INK4a and p21Cip1, can be determined by immunochemistry methods practiced in the art, such as immunoblotting analysis (Dimri, et al. 1996 Biol. Signals 5:154-162). Expression of p16 mRNA in a cell can be measured by techniques practiced in the art including quantitative PCR. The presence and level of senescence cell associated polypeptides (e.g., polypeptides of the SASP, generally called SASP factors or proteins, or senescence messaging secretome (SMS) can be determined by using automated and high throughput assays. The presence of senescent cells can also be determined via detection of senescent cell-associated molecules, which include growth factors, proteases, cytokines (e.g., inflammatory cytokines), chemokines, cell-related metabolites, reactive oxygen species (e.g., H₂O₂), and other molecules that stimulate inflammation and/or other biological effects or reactions that may promote or exacerbate the underlying disease of the subject.

### Long-Term Expansion of Stem Cells Leads to Cellular Senescence

Stem cell therapies have traditionally utilized in vitro expansion in order to increase cell numbers, based on the presumption that hundreds of millions of cells are necessary for therapeutic efficacy. However, it is described herein that long-term expansion of stem cells leads to the accumulation of senescent cells (Example 1). The senescence-associated secretory phenotype (SASP) can adversely affect not only neighboring cells, but also likely contribute to driving systemic aging. Transplantation of senescent cells not only can induce an osteoarthritis-like condition in mice, but the senescent cells caused leg pain, impaired mobility, and radiographic and histological changes suggestive of OA (Xu, et al. 2017 J Gerontol A Biol Sci Med Sci 72(6):780-785). Avoiding long-term expansion when utilizing stem cells for treating OA is advantageous, because senescent cells produced by expansion would adversely affect efficacy of adult stem cells for articular cartilage (AC) repair. BMSCs from bone marrow aspirate concentrate (BMC) offer a significant advantage over other stem cell therapies, because these cells do not require in vitro expansion, which would likely increase the number of senescent cells and consequently reduce their therapeutic benefits for treating OA.

### Senolytic Agents

Senolytic agents are agents that selectively target and induce apoptosis/death of senescent cells (Kirkland, et al. 2017 J Am Geriatr Soc 65(10):2297-2301; Zhu, et al. 2015 Aging Cell 14(4):644-658). Senolytic agents include, without limitation, flavonoids (quercetin, Fisetin), tyrosine kinase inhibitor (e.g., dasatinib)+quercetin, alkanoids (piperlongumine), curcumin analog, navitoclax, 17-DMAG, BCL-2-targeting agents (ABT-263, ABT-737), and combinations thereof. Specifically, these agents target senescent cell anti-apoptotic pathways (SCAPs), which are upregulated during senescence. Senolytic agents are sometimes included in a group of interventions known as “geroprotectors” or “senotherapies”. Senolytic agents also include geroprotective nutrients such as, without limitation, myricetin, N-acetyl-cysteine (NAC), gamma tocotrienol, or epigallocatechin-gallate (EGCG).

Fisetin is a natural flavonoid found in many fruits and vegetables. It is a known antioxidant and reducing agent due to its hydroxyl groups. It has been shown to reduce the secretion of several proinflammatory factors and has anti-cancer activity, blocking the mTOR and PI3K/AKT pathway, making Fisetin a strong therapeutic for targeting senescent cells. Fisetin has the molecular formula C₁₅H₁₀O₆, molecular weight 286.24 g/mol, CAS name/number: Fisetin, 2-(3,4-dihydroxyphenyl)-3,7-dihydroxychromen-4-one, 528-48-3, and chemical structure:

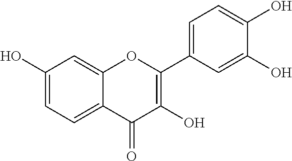 [SMILES: O=c1c(O)c(-c2ccc(O)c(O)c2)oc2cc(O)ccc12]

Senolytic drugs like Fisetin can effectively and selectively eliminate senescent cells, thus offering a safe and novel therapeutic strategy for the reduction of senescence in orthobiologics.

### Anti-Fibrotic Agents

Fibrocartilage is the predominant repair tissue following a cartilage repair procedure or stem cell transplantation. TGF-β1-pSmad2/3 signaling plays a major role in tissue fibrosis (Li, et al. 2004 Am J Pathol 164(3):1007-19). In addition, several studies have shown that blocking TGF-β1 can inhibit tissue fibrosis and improve musculoskeletal tissue repair. Blocking TGF-β1 may be a good strategy to limit fibrocartilage (fibrosis in cartilage) and enhance hyaline-like cartilage. However, TGF-β1 is widely believed to be essential for articular cartilage homeostasis and repair (Zhen and Cao 2014 Trends Pharmacol Sci 35(5):227-36). Although it has been reported that the lack of TGF-β1 results in osteoarthritis, it has also been shown that TGF-β1 is overproduced in osteoarthritic joints. Preliminary data indicate a benefit to cartilage repair with microfracture when Losartan, a TGF-β1 antagonist, is added to the microfracture (Utsunomiya, et al. 2019 Orthopaedic J Sports Med 7(7_supp15): p. 2325967119S00263). Hence, disclosed herein is combining Losartan with orthobiologics, for example, with BMSCs to improve the beneficial effect of BMSCs on AC repair after OA.

Anti-fibrotic agents contemplated herein include, without limitation, angiotensin 1 receptor antagonists (for example, Losartan), TGF-β1 receptor antagonists (for example, Suramin), γ-interferon, TGF-β1 antagonists (for example, Pirfenidone), TGF-β1 ligand binders (for example, Decorin), and Halofuginone. MMP treatment (for example, MMP-1, MMP-3, MMP-9) is also contemplated herein for its reduction of muscle fibrosis after injury.

Losartan is an angiotensin II receptor antagonist. It is used to treat high blood pressure (hypertension) and to reduce the risk of a stroke and works by constricting blood vessels. Its CAS number is 114798-26-4, and its chemical structure is:

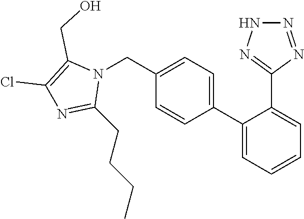 [SMILES: CCCCc1nc(Cl)c(CO)n1Cc1ccc(-c2ccccc2-c2nn[nH]n2)cc1]

Losartan is also an anti-fibrotic agent that also improves both muscle regeneration and function in several models of recoverable skeletal muscle injuries.

### Methods for Enhancing a Therapeutic Outcome

The present disclosure includes methods for enhancing a therapeutic outcome in a subject, comprising administering at least one senolytic agent and/or at least one anti-fibrotic agent to the subject.

As used herein, the terms “enhancing”, “improving”, “ameliorating”, or the like, mean to alleviate symptoms, eliminate the causation of symptoms either on a temporary or permanent basis, or to prevent or slow the appearance of symptoms of a musculoskeletal condition or disorder in a subject. In certain embodiments, the musculoskeletal condition includes slow healing, including after injury (wound healing) or surgery (post-procedure healing, response to physical therapy).

In specific embodiments of a method according to the invention, the therapeutic outcome is related to the outcome of surgical and/or non-surgical treatment of a musculoskeletal disorder. In further embodiments, the therapeutic outcome is related to the outcome of surgical and/or non-surgical treatment of a bone injury or bone condition or bone disorder.

In specific embodiments of a method according to the invention, the enhanced therapeutic outcome comprises further delaying the onset of osteoarthritis.

An improvement in a surgical outcome (in the case of surgery as the treatment) means a positive change from baseline. In this context, the term “positive” refers to a change associated with better healing or other clinical outcomes such as improved pain scores or mobility. For example, healing time is reduced, mobility is increased (for example, mobility of a joint is improved, as assessed by functional performance testing), cartilage of a joint is improved (as assessed by MRI, T2 mapping, or the like), pain is reduced (as assessed by PROs), scar tissue is reduced, and/or there is enhanced healing of soft tissue (i.e., following ACLR procedure), and/or senescence markers are reduced. As used herein, the term “baseline” means the numerical value of the parameter for a subject prior to or at the time of treatment according to the present invention.

To determine whether the surgical outcome/parameter has “improved,” the parameter is quantified at baseline and at one or more time-points after treatment. The difference between the value of the parameter at a particular time point following initiation of treatment and the value of the parameter at baseline is used to establish whether there has been an “improvement”.

In certain embodiments, the surgical treatment is an operative treatment for articular cartilage pathology falling into one of three main categories: i) articular surface debridement, ii) autologous chondrocyte implantation (ACI), and iii) total joint replacement. Surface debridement is generally ineffective for treating OA. ACI requires multiple surgeries and has shown mixed results for delaying OA progression. Total joint replacement is not an ideal treatment option, especially for younger patients. Thus, there is a significant need for biologically driven therapies that can preserve and/or restore articular cartilage.

In certain embodiments, the non-surgical treatment comprises administration of an orthobiologic to the subject. In on embodiment, the non-surgical treatment comprises administration of bone marrow stem cells to a subject for the treatment of osteoarthritis.

To determine whether a non-surgical outcome/parameter has “improved,” the parameter is quantified at baseline and at one or more time-points after treatment. The difference between the value of the parameter at a particular time point following initiation of treatment and the value of the parameter at baseline is used to establish whether there has been an “improvement”.

For example, healing time is reduced, mobility is increased (for example, mobility of a joint is improved, as assessed by functional performance testing), cartilage of a joint is improved (as assessed by MRI, T2 mapping, or the like), pain is reduced (as assessed by PROs), scar tissue is reduced, and/or there is enhanced healing of soft tissue, and/or senescence markers are reduced.

OA knee joints can, in specific embodiments, undergo MRI at baseline, 6 months, and 18 months post-treatment to assess changes in cartilage morphology and structure over time. Patient-reported outcomes for pain and function can be collected at baseline and 3, 6, 12 & 18 months. Joint and cartilage function can be assessed using video-motion analysis at baseline, 6 months, and 18 months to assess joint kinematics and kinetics. OA biomarkers related to cartilage degeneration, inflammation and pain can be assessed at baseline and 18 months. Blood and synovial fluid, as well as patient-reported outcomes, can be collected throughout the study described herein, including at baseline, 4 days, and 18 months after treatment to assess changes in cellular senescence and OA biomarkers, and to assess pain and function related to cartilage degeneration and inflammation.

Musculoskeletal Conditions and/or Disorders

Musculoskeletal conditions and/or disorders include injuries and disorders that affect the body's movement or musculoskeletal system and include injuries or disorders of the muscles (for example, sarcopenia, fibromyalgia), nerves, tendons, joints (for example, osteoarthritis, rheumatoid arthritis, psoriatic arthritis, gout, ankylosing spondylitis), bones (osteoporosis, osteopenia and associated fragility fractures, traumatic fractures), cartilage, spine, and spinal discs. Common musculoskeletal disorders affecting the muscles, bones, and/or joints include tendonitis, carpal tunnel syndrome, osteoarthritis, rheumatoid arthritis, and bone fractures.

In specific embodiments of a method according to the invention, the musculoskeletal condition or disorder is osteoarthritis. In further specific embodiments of a method according to the invention, the musculoskeletal condition or disorder is an articular cartilage defect. The healing potential of articular cartilage (AC) is extremely limited, because adult articular cartilage exhibits neither vascularization nor innervation, and defects larger than 2-4 mm in diameter rarely heal. A healing-related inflammatory response occurs only when full-thickness articular cartilage defects also injure the subchondral bone. Unfortunately, even under such circumstances, the regenerated tissue is fibrocartilage, which is histologically dissimilar and biomechanically inferior to native, hyaline cartilage. Articular cartilage injuries may result from trauma, but the most common cause of articular cartilage damage is osteoarthritis (OA).

As used herein, the expression “a subject in need thereof” means a human or non-human mammal that exhibits one or more symptoms or indications of a musculoskeletal condition or disorder, and/or who has been diagnosed with a musculoskeletal condition or disorder. Throughout the present disclosure, the terms “subject”, “patient”, and “subject in need thereof” are used interchangeably. The term “a subject in need thereof” may also include a patient who is going to undergo treatment and/or surgery, for example, orthopedic surgery. The term “a subject in need thereof” may also include, e.g., patients who, prior to treatment, have measurable senescence/senescent cells, senescence biomarkers, and/or SASP in a sample obtained from the subject. In some embodiments, the sample is selected from the group consisting of peripheral blood mononuclear cells (PBMCs), plasma, serum, bone marrow, marrow-derived plasma, synovial cells, and synovial fluid.

### Pharmaceutical Compositions

The present disclosure includes methods that comprise administering at least one senolytic agent and/or at least one anti-fibrotic agent to a subject, wherein each agent is contained within a pharmaceutical composition. The pharmaceutical compositions for use according to the disclosure may be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical compositions for use according to the disclosure, e.g., a bioengineered scaffold, encapsulation in liposomes, microparticles, microcapsules, recombinant cells capable of expressing the mutant viruses, receptor mediated endocytosis (see, e.g., Wu et al., 1987, J. Biol. Chem. 262: 4429-4432). Methods of administration include, but are not limited to, intradermal, intra-articular, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In a preferred embodiment, the composition is administered orally.

In certain situations, the pharmaceutical composition can be delivered in a controlled release system. In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

Injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the agent in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc.

Pharmaceutical compositions for oral or parenteral use are prepared into dosage forms in a unit dose suited to fit a dose of the active ingredients. Such dosage forms in a unit dose include, for example, tablets, pills, capsules, injections (ampoules), suppositories, etc.

### Administration Regimens

The senolytic agent (for example, Fisetin) can either be applied directly to the orthobiologic (for example, bone marrow aspirate concentrate (BMC) or delivered systemically (via oral dosing) for improving the orthobiologic indirectly. The use of an oral dietary supplement minimizes the burden for regulatory approval. Furthermore, studies show that Fisetin treatment, like other related senolytic agents, requires only intermittent dosing to be effective, given that the accumulation of detectable senescent cells takes weeks to occur (Zhu, et al. 2015 Aging Cell 14(4):644-58). Thus, dosing regimens at weekly or even monthly frequencies are significantly more manageable and sustainable for the patient with significantly less chance for potential side-effects.

The present disclosure includes methods comprising administering to a subject at least one senolytic agent at a dosing frequency of at least once. In additional embodiments, the disclosed methods comprise administering to a subject at least one senolytic agent at a dosing frequency of more than once. In still further embodiments, dosing is such that a therapeutic response is achieved. The therapeutic response in this context constitutes a reduction in senescent cells. In one embodiment, a senolytic agent is administered for at least three months before treatment, for at least two months before treatment, or for at least one month before treatment. In another embodiment, a senolytic agent is administered orally twice per month for one month before treatment. In a further embodiment, Fisetin is administered orally 2 daily doses back-to-back, followed by 28 days off, before treatment.

The present disclosure additionally includes methods comprising administering to a subject at least one senolytic agent at a dosing frequency of more than once, at least once before treatment and at least once after treatment. The therapeutic response in this context constitutes a reduction in senescent cells. In one embodiment, a senolytic agent is administered for at least three months before treatment, for at least two months before treatment, or for at least one month before treatment, and is administered for at least one month after treatment, for at least two months after treatment, or for at least three months after treatment. In another embodiment, a senolytic agent is administered orally twice per month for one month before treatment and twice per month for one month after treatment. In a further embodiment, Fisetin is administered orally 2 daily doses back-to-back, followed by 28 days off, before treatment and 2 daily doses back-to-back, followed by 28 days off after treatment.

The present disclosure includes methods comprising administering to a subject at least one senolytic agent and at least one anti-fibrotic agent at a dosing frequency of at least once each. In additional embodiments, the disclosed methods comprise administering to a subject at least one senolytic agent and at least one anti-fibrotic agent at a dosing frequency of more than once each. In still further embodiments, dosing is such that a therapeutic response is achieved. The therapeutic response in this context constitutes a reduction in senescent cells and a reduction in fibrosis. In one embodiment, a senolytic agent is administered for at least three months, for at least two months, or for at least one month before treatment, and an anti-fibrotic agent is administered for at least one month, for at least two months, for at least three months, for at least four months, for at least five months, or for at least six months after treatment. In another embodiment, Fisetin is administered (for example, orally) twice per month for one month before treatment, and Losartan is administered (for example, orally) for up to six months after treatment. In a further embodiment, Fisetin is administered orally 2 daily doses back-to-back, followed by 28 days off, before treatment, and Losartan is administered daily for up to six months after treatment. In still a further embodiment, Fisetin is administered orally 2 daily doses back-to-back, followed by 28 days off, before treatment, and Losartan is administered daily for up to three months after treatment.

The present disclosure additionally includes methods comprising administering to a subject at least one senolytic agent at a dosing frequency of more than once, at least once before treatment and at least once after treatment, and at least one anti-fibrotic agent at a dosing frequency of at least once after treatment. The therapeutic response in this context constitutes a reduction in senescent cells and a reduction in fibrosis. In one embodiment, a senolytic agent is administered for at least three months, for at least two months, or for at least one month before treatment and for at least one month, for at least two months, or for at least three months after treatment, and an anti-fibrotic agent is administered for at least one month, for at least two months, for at least three months, for at least four months, for at least five months, or for at least six months after treatment. In another embodiment, a senolytic agent is administered orally twice per month for at least one month before treatment and twice per month for at least one month after treatment, and an anti-fibrotic agent is administered orally for up to six months after treatment. In another embodiment, a senolytic agent is administered orally twice per month for one month before treatment and twice per month for one month after treatment, and an anti-fibrotic agent is administered orally for up to three months after treatment. In still a further embodiment, Fisetin is administered orally 2 daily doses back-to-back, followed by 28 days off, before treatment and 2 daily doses back-to-back, followed by 28 days off after treatment, and Losartan is administered daily for up to three months after treatment.

The present disclosure additionally includes methods comprising administering to a subject at least one senolytic agent at a dosing frequency of more than once, at least once before treatment and at least once after treatment, and at least one anti-fibrotic agent at a dosing frequency of more than once, at least once before treatment and at least once after treatment. The therapeutic response in this context constitutes a reduction in senescent cells and a reduction in fibrosis.

In certain embodiments of a method according to the disclosure, the at least one senolytic agent and the at least one anti-fibrotic agent are administered singly to the subject. “Singly”, as used herein, refers to the agents being administered to the subject at separate times. In further embodiments, the at least one senolytic agent and the at least one anti-fibrotic agent are administered concomitantly to the subject. The clinician is mindful of possible drug interactions and side effects. “Drug” or “therapeutic agent”, as used herein, includes supplements, including dietary supplements. The specific indication (musculoskeletal condition or disorder) may also dictate the administration regimen (including dosages) of the senolytic agent and the anti-fibrotic agent.

According to certain embodiments of the present disclosure, multiple doses of at least one senolytic agent may be administered to a subject over a defined time course. The methods according to this aspect of the disclosure comprise sequentially administering to a subject multiple doses of the agent. As used herein, “sequentially administering” means that each dose of the agent is administered to the subject at a different point in time, e.g., on different days separated by a predetermined interval (e.g., hours, days, weeks or months). The sequentially administered doses may all contain the same amount of agent, but generally may differ from one another in terms of frequency of administration. In certain embodiments, however, the amount of agent contained in the sequentially administered doses varies from one another (e.g., adjusted up or down as appropriate).

The methods of the present disclosure, according to certain embodiments, comprise administering to the subject an additional therapeutic agent in combination with the at least one senolytic agent and/or at least one anti-fibrotic agent. As used herein, the expression “in combination with” means that the additional therapeutic agent is administered before, after, or concurrent with the senolytic agent and/or anti-fibrotic agent. The term “in combination with” also includes sequential or concomitant administration of the senolytic agent and/or anti-fibrotic agent and the additional therapeutic agent.

### Dosage

The amount of the at least one senolytic agent administered to a subject according to the methods of the present invention is, generally, a therapeutically effective amount. As used herein, the phrase “therapeutically effective amount” means an amount of senolytic agent that results in one or more of: (a) a measurable reduction in senescent cells; and/or (b) an improvement in a symptom of a musculoskeletal condition or disorder.

A therapeutically effective amount of a senolytic agent can be from about 0.05 mg to about 1000 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, about 2000 mg, about 2050 mg, about 2100 mg, about 2150 mg, about 2200 mg, about 2250 mg, about 2300 mg, about 2350 mg, about 2400 mg, about 2450 mg, about 2500 mg, about 2550 mg, about 2600 mg, about 2650 mg, about 2700 mg, about 2750 mg, about 2800 mg, about 2850 mg, about 2900 mg, about 2950 mg, about 3000 mg, about 3050 mg, about 3100 mg, about 3150 mg, about 3200 mg, about 3250 mg, about 3300 mg, about 3350 mg, about 3400 mg, about 3450 mg, about 3500 mg, about 3550 mg, about 3600 mg, about 3650 mg, about 3700 mg, about 3750 mg, about 3800 mg, about 3850 mg, about 3900 mg, about 3950 mg, about 4000 mg, about 4050 mg, about 4100 mg, about 4150 mg, about 4200 mg, about 4250 mg, about 4300 mg, about 4350 mg, about 4400 mg, about 4450 mg, about 4500 mg, about 4550 mg, about 4600 mg, about 4650 mg, about 4700 mg, about 4750 mg, about 4800 mg, about 4850 mg, about 4900 mg, about 4950 mg, about 5000 mg, or any amount inbetween, of the senolytic agent. In certain embodiments, the at least one senolytic agent is Fisetin, and it is administered at a dosage of 1000 mg/day.

In another embodiment, the at least one senolytic agent is Fisetin, and it is administered at a dosage of about 10 mg/kg/day to about 100 mg/kg/day. In another embodiment, the at least one senolytic agent is Fisetin, and it is administered at a dosage of about 10 mg/kg/day to about 50 mg/kg/day. In yet another embodiment, the at least one senolytic agent is Fisetin, and it is administered at a dosage of about 20 mg/kg/day.

The amount of the at least one anti-fibrotic agent administered to a subject according to the methods of the present invention is, generally, a therapeutically effective amount. As used herein, the phrase “therapeutically effective amount” means an amount of anti-fibrotic agent that results in one or more of: (a) a reduction in fibrosis; and/or (b) an improvement in a symptom of a musculoskeletal condition or disorder. Minimizing further fibrosis is also considered a reduction in fibrosis herein. A reduction in fibrosis may, in certain embodiments, refer to a reduction in fibrotic sequelae in various musculoskeletal tissues including, without limitation, muscle, bone, and/or cartilage. A reduction in fibrosis may, in additional embodiments, refer to a reduction in “scarring” and/or an increase in tissue reserve or function, as it relates to musculoskeletal biomechanics or biology.

A therapeutically effective amount of an anti-fibrotic agent can be from about 0.05 mg to about 1000 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, or any amount inbetween, of the anti-fibrotic agent. In certain embodiments, the at least one anti-fibrotic agent is Losartan, and it is administered at a dosage of about 10 mg/day to about 200 mg/day. In further embodiments, the at least one anti-fibrotic agent is Losartan, and it is administered at a dosage of about 10 mg/day to about 100 mg/day. In further embodiments, the at least one anti-fibrotic agent is Losartan, and it is administered at a dosage of about 10 mg/day to about 50 mg/day. In still further embodiments, the at least one anti-fibrotic agent is Losartan, and it is administered at a dosage of 25 mg/day.

### Orthobiologics

The term “orthobiologics” or “orthobiologic products”, as used herein, refers to biologic substances that are used to improve healing of bone, cartilage, tendon, and/or ligament, for example, after injury or surgery. The products are deemed biologic, because they are made from substances naturally found in the body. Orthobiologics are advantageous in that they minimize the impact of degenerative disease and allow for more rapid recovery from musculoskeletal injury.

Orthobiologics typically include bone grafts, autologous blood, platelet-poor plasma (PPP), platelet-rich plasma (PRP), including derivatives with high or low leukocyte content (LR-PRP, LP-PRP), autologous conditioned serum, bone marrow aspirate concentrate (BMAC), and autologous stem cells, including mesenchymal stem cells (MSCs) and adipose-derived stem cells (ASCs). Orthobiologics may also include agents such as anti-fibrotic agents, senotherapeutics, fat grafts like microfragmented adipose tissue, nanofragmented adipose tissue, product derived from birth tissues, Extra cellular matrix (ECM) implants, supplements, alpha-2-macroglobulin (A2M), amniotic fluid, placental tissue, umbilical cord tissue, hyaluronic acid injections (or other viscosupplements), and stem cell injections. Contemplated stem cells include, without limitation, ADSCs, ex vivo culture-expanded stem cells, freshly isolated stem cells, endogenous stem cells, bone marrow aspirate concentrate (BMAC) stem cells, and whole blood stem cells.

The orthobiologic product may be selected, without limitation, from the group consisting of a bone graft, autologous blood, platelet-rich plasma (PRP), autologous conditioned serum, stem cells, Bone Marrow Aspirate Concentrate (BMAC), Platelet Rich Plasma (PRP) PRP, Alpha 2 Macroglobulin (A2M), amniotic fluid, placental tissue, umbilical cord tissue, and hyaluronic acid.

Adult stem cells are more attractive than primary chondrocytes because of their availability, capacity for self-renewal, high proliferative capacity, and multipotency. Several studies have demonstrated that stem cells can undergo chondrogenesis and repair AC in experimentally induced cartilage injury models (osteochondral lesions). Although stem cell-based therapies have already been used clinically for cartilage repair (Wakitani, et al. 2004 Cell Transplant 13(5):595-600; Kuroda, et al. 2007 Osteoarthritis Cartilage 15(2):226-31), the success remains limited. Thus, the development of novel strategies to increase the regenerative potential of adult stem cells for maintaining or improving articular cartilage health in knees with OA are disclosed herein.

Joint injection of bone marrow-derived stem cells (BMSCs), typically performed using autologous bone marrow aspirate concentrate (BMC), has shown efficacy for reducing knee OA symptoms with minimal adverse effects, but study results are mixed and hard to compare due to small sample sizes, lack of rigorous study designs, variable preparations, inconsistent outcome measures, and short follow-up post-treatment (Jevotovsky, et al. 2018 Osteoarthritis Cartilage 26(6):711-729). Furthermore, while cohort studies and retrospective analyses have shown reduction of pain up to 1 year after BMC injection, clinical results of BMC injection are highly variable across patients. It also remains unclear whether a single injection containing BMC alone can actually modify disease progression or provide only relatively short-term pain relief (Di Matteo, et al. 2019 Stem Cells Int p. 1735242).

Bone marrow concentrate (BMC) contains a variety of cells including bone marrow mesenchymal stem cells, hematopoietic stem cells (HSCs), perivascular cells (PVs), and endothelial cells (ESCs). BMC is stem cell therapy that relies upon a natural combination of different stem cell populations residing in bone marrow with potentially unaltered niches that has been proven safe and effective for the treatment of OA-related symptoms (Chahla, et al. 2017 Arthrosc Tech 6(2):e441-e445; Chahla, et al. 2016 Orthop J Sports Med 4(1):2325967115625481). It is generally thought that resident stem cells, such as multipotent mesenchymal stem cells, are a significant contributor of the pro-regenerative effects of bone marrow treatments through their ability to differentiate into various tissue and secrete tissue repairing factors. However, recent evidence suggests that utilizing the complete bone marrow niche, comprised of several cell types as well as naturally occurring growth factors and cytokines, may be more advantageous than engineered or synthetic treatment options or isolated and expanded stem cells for treating OA. It is suggested herein that optimization of BMC to improve its clinical efficacy may be needed in order to target disease modifying processes, as opposed to treatment of OA-related symptoms. Targeting aged or senescent cells that have been shown to contribute to OA onset and symptoms is, thus, contemplated herein.

Thus, in one embodiment, the orthobiologic is bone marrow-derived stem cells (BMSCs)/bone marrow aspirate concentrate. Bone marrow concentrate represents a highly translationally relevant source of stem cells (bone marrow-derived) with established bioactivity and capacity for chondrogenic differentiation (Hindle, et al. 2017 Stem Cells Transl Med 6(1):77-87). “Bone marrow aspirate concentrate” and “bone marrow concentrate” are used interchangeably herein.

Senescent HSCs and MSCs isolated from bone marrow of aged humans have been shown to be dysfunctional, exhibit pro-inflammatory phenotypes, and evidence of DNA damage. However, age-associated changes in senescent cell number and/or senescence associated markers in BMC ex vivo have yet to be investigated. Furthermore, several studies have identified T-Cells as strong correlates to chronological age and health status (Liu, et al. 2009 Aging Cell 8(4):439-48). Thus, these cells can serve as strong cellular indicators of senescent burden in BMSCs. The assessment of quantifiable levels of senescent cells (CD3+ T-Cells), senescence associated transcripts, and senescence associated secretory factors from samples of BMC at different ages showed that the number of senescent cells and SASP in BMC increased with age (Example 1). Hence, combining senolytic agents with BMSCs may improve the beneficial effect of BMSCs on AC repair after OA.

In one embodiment, Fisetin treatment can be added to orthobiologics for patients with moderate osteoarthritis of the knee and/or hip, decreasing patient-reported pain and cartilage loss relative to patients that do not receive senolytic therapies with orthobiologics.

### C₁₂FDG

The use of flow cytometry-assisted analysis of senescent peripheral blood mononuclear cells for clinical diagnostic use often relies on certain subsets of Peripheral Blood Mononuclear Cells (PBMCs), namely T-Cells, as they are best suited to reflect chronological age or senescence state in human whole blood. However, there is debate as to which subset of T-Cells might best reflect senescent state (i.e., CD4 vs CD8, etc.). Selecting all T-Cells (CD3+), including both CD4 and CD8 subsets from PBMCs, enables detection of reproducible and age-correlative changes in senescent cell number using C₁₂FDG staining.

C₁₂FDG (5-Dodecanoylaminofluorescein Di-β-D-Galactopyranoside) is the β-galactosidase substrate that is covalently modified to include a 12-carbon lipophilic moiety. Once inside the cell by staining procedure, the substrate is cleaved by β-galactosidase enzyme, producing a fluorescent product that is well retained by the cells, probably by incorporation of the lipophilic tail within the cell membrane. Thus, C₁₂FDG is used as a fluorescent stain, as opposed to involving antibody staining immunophenotyping. Using benchtop flow cytometry, the senescent cells assay can be run and completed in a matter of hours and with a minimum amount of blood (as little as 5 ml). The detection and measurement of senescent cells using C₁₂FDG is disclosed in US2021/0046123A1, incorporated herein in its entirety.

In one embodiment, a method according to the disclosure further comprises detecting and/or measuring senescent cells in a sample (from a subject), comprising staining the sample cells with C₁₂FDG; and subjecting the stained cells to flow cytometry. The sample may be selected from the group consisting of, but not limited to, peripheral blood mononuclear cells (PBMCs), plasma, serum, bone marrow, marrow-derived plasma, synovial cells, and synovial fluid. In still another embodiment, the detected senescent cells are characterized according to stage of senescence. In still another embodiment, the characterization is based on brightness of signal. In a further embodiment, the stage of senescence is early-stage (relatively low C₁₂FDG positivity, “dim”, low green fluorescent intensity on a flow cytometry plot), mid-stage (relatively moderate C₁₂FDG positivity), or late-stage (relatively high C₁₂FDG positivity, “bright”, high fluorescent intensity on a flow cytometry plot), as determined by normalized event gating with flow cytometry. In certain embodiments, the late-stage senescent cells are the target of the at least one senolytic agent.

### Kits

Additionally provided herein are kits for carrying out the methods according to the disclosure. The kits comprise at least one senolytic agent and/or at least one anti-fibrotic agent. In certain embodiments, the kits further comprise instructions for use. The instructions may be in a tangible form. In additional embodiments, the kits include materials and/or instructions for assessment of an enhanced therapeutic outcome.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.), but some experimental errors and deviations should be accounted for. Unless indicated otherwise, parts are parts by weight, molecular weight is average molecular weight, temperature is in degrees Centigrade, and pressure is at or near atmospheric.

### Example 1. Eliminating Senescent Cells can Improve the Effect of BMSC Treatment for Knee OA

### Injection of Senescent Cells Induced OA-Like Conditions in the Knee Joint.

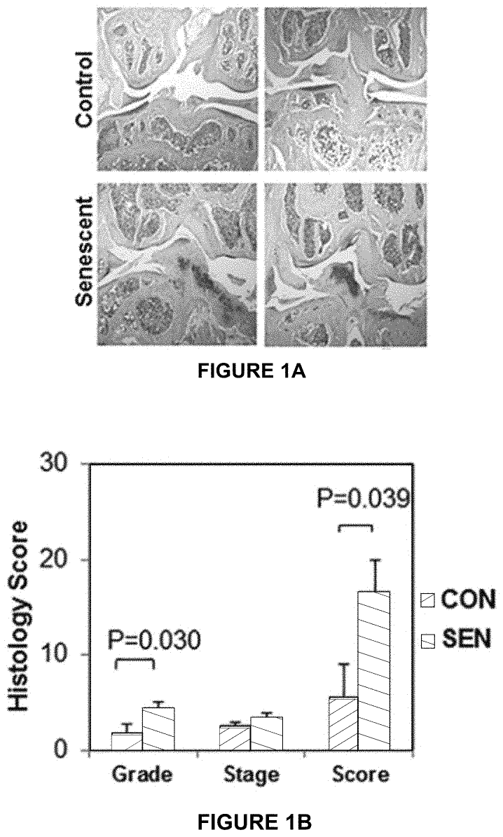

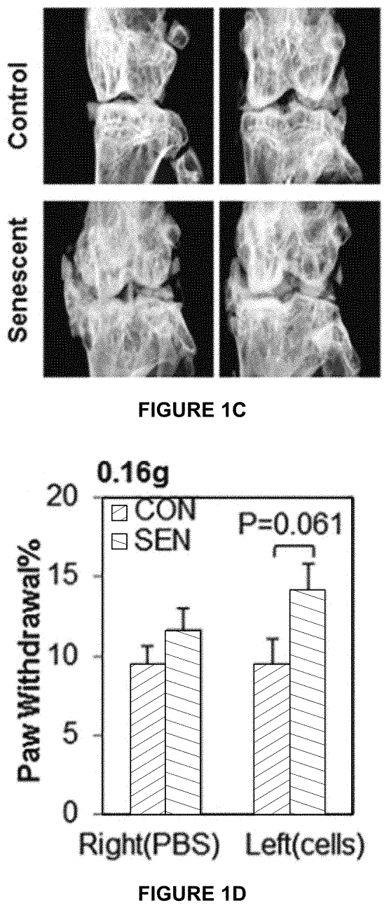

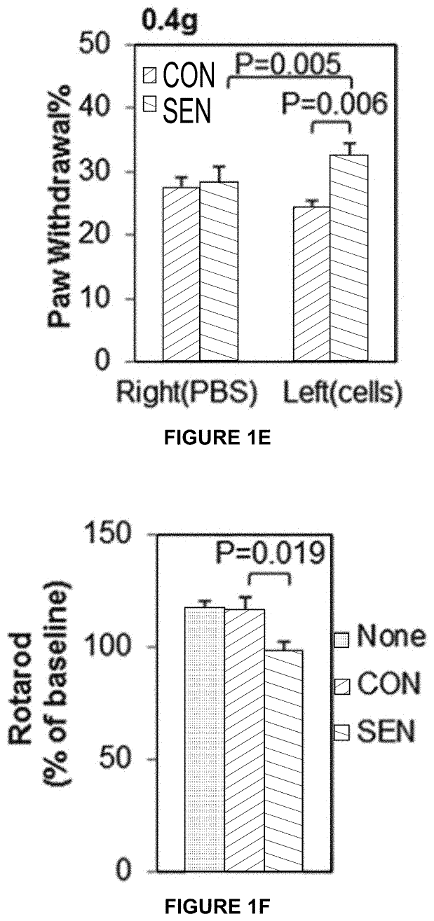

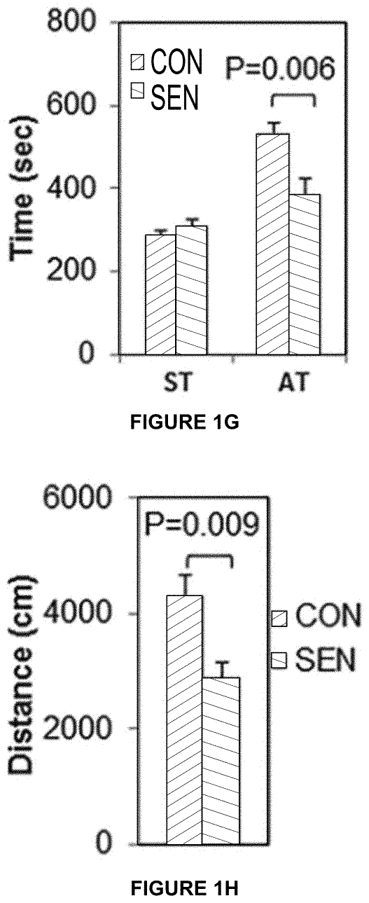

A small number of senescent or non-senescent cells from the ear cartilage of luciferase-expressing mice were injected into the knee joint area of wild-type mice (Xu, et al. 2017 J Gerontol A Biol Sci Med Sci 72(6):780-785). Injected cells were tracked in vivo for more than 10 days using bioluminescence and ¹⁸FDG PET imaging. 7-month-old C57BL/6 female mice were subjected to non-senescent (CON) or senescent (SEN) primary ear chondrocytes transplanted into the knee (FIG. 1A). Safranin 0/Fast Green staining was performed 3 months after transplantation, and histology scores and representative radiographs are shown (FIGS. 1 B, 1C). Pain and grip strength were assessed using von Frey filament assays 3 months post transplantation (FIGS. 1 D, 1E) and Rotarod assays before and 1 month after transplantation (FIG. 1F). Locomotor activity during 20 minutes of evaluation was monitored 3 months after transplantation (FIGS. 1G, 1H) These results taken together indicate that transplanting senescent cells into the knee caused leg pain, impaired mobility, and OA-like radiographic and histological changes. Thus, targeting senescent cells could be a promising strategy for treating OA. In fact, the elimination of senescent cells can extend healthspan and prevent the development of multiple age-associated morbidities including OA. It is, thus, evaluated herein whether senolytic drugs can eliminate senescent cells and improve outcomes with BMSC treatment for OA.

Presence of Senescent Cells and SASPs Increase with Age in Bone Marrow.

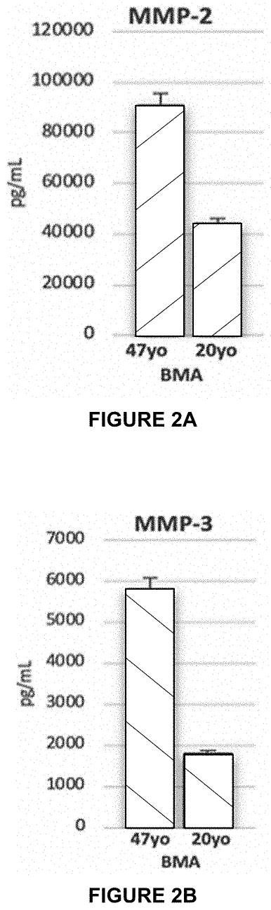

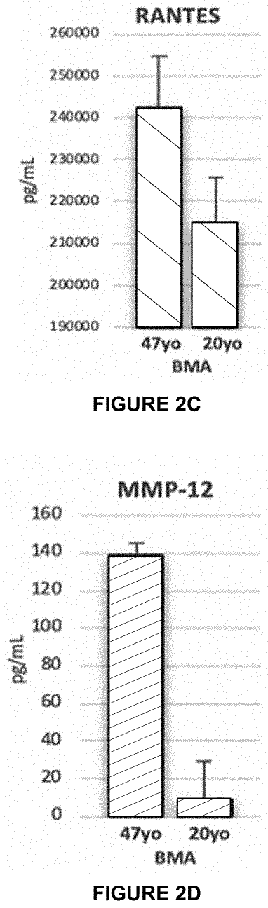

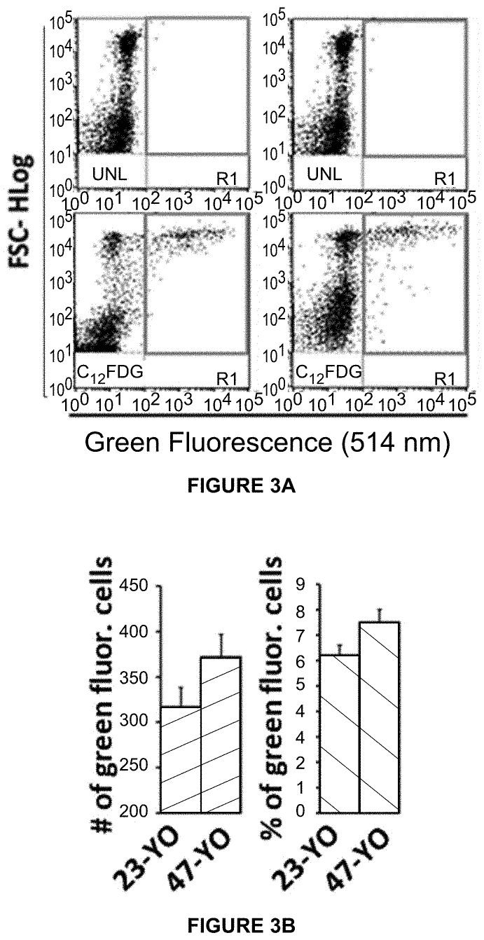

Proof-of-concept experiments were performed to characterize age-related deviations in senescence markers in bone marrow aspirate (BMA) from samples collected from two female osteoarthritis patients, ages 23 and 47. Following gradient centrifugation, plasma was collected and CD3⁺ T-Cells were enriched from PBMCs. BMA levels of several SASP factors (MMP-2,3,12 and RANTES) were found to increase significantly with age, suggesting that the number of senescent cells increased with age as well (FIGS. 2A-2D). Indeed, using flow cytometry and the fluorescent senescent cell label C₁₂FDG, the number of senescent CD3+ T-Cells increased with age, even when the age difference was only 24 years (FIGS. 3A and 3B). In peripheral blood CD3⁺ T-Cells from a young (18 y.o.) and a late-aged donor (82 y.o.), an even more exaggerated increase was observed in senescent cells using C₁₂FDG staining (18 y.o., 8.1%; 82 y.o., 28.0%). The ability to properly characterize the senescent profile of BMA and peripheral blood using several modalities allows the assessment of contributions of senescent cells and their SASP factors to OA symptoms and the determination of the beneficial effects of BMSC for treating knee OA.

### Senolytic Drug can Eliminate Senescent Cells in Adult Stem Cell Populations.

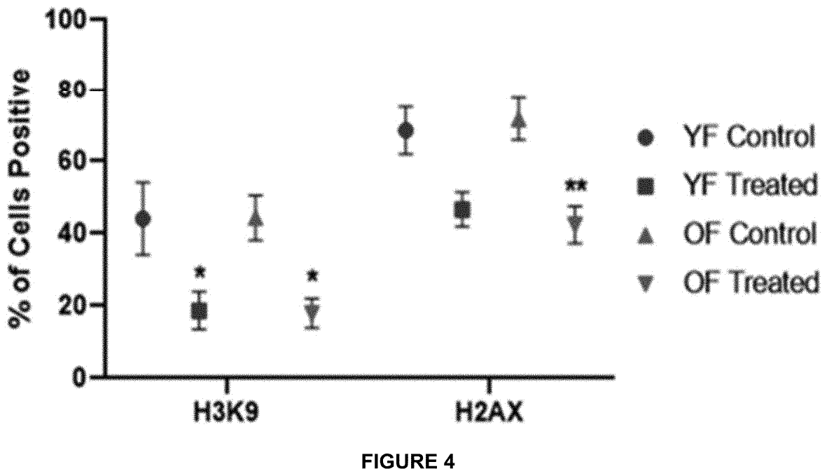

In preliminary experiments (FIG. 4), pre-existing senescent cells were found in cultured human adipose-derived stem cells (ADSCs). The ability of these cells to produce SASPs can induce significant damage to other stem cells or endogenous cells once re-administered into the intra-articular space. Thus, avoiding the proliferation of senescent cells from long-term expansion is critical to prevent these cells from compromising the ability of adult stem cells to repair cartilage. The number of senescent cells in culture-expanded ADSCs can be significantly reduced by adding the senolytic agent Fisetin. ADSCs from a young female (27 years old, YF) and old female (79 years old, OF) were cultured to passage 4 using normal proliferation media (DMEM/F-12, 10% FBS, 1% penicillin/streptomycin). The cells were subsequently treated with 50 μM of Fisetin for 24 hours and then cultured for 48 hours. Senescent cells were identified using antibodies for senescence-associated heterochromatin foci. Fisetin significantly reduced the number of senescent cells in the expanded ADSCs in vitro (FIG. 4). These experiments support that Fisetin can eliminate senescent cells and SASPs in expanded stem cell populations, and this may apply to harvested tissues such as BMC from older OA patients that likely contain senescent cells (see FIGS. 2A-4, above).

### Senolytic Treatment Delays OA Symptoms in Progeroid Z24−/− Mice.

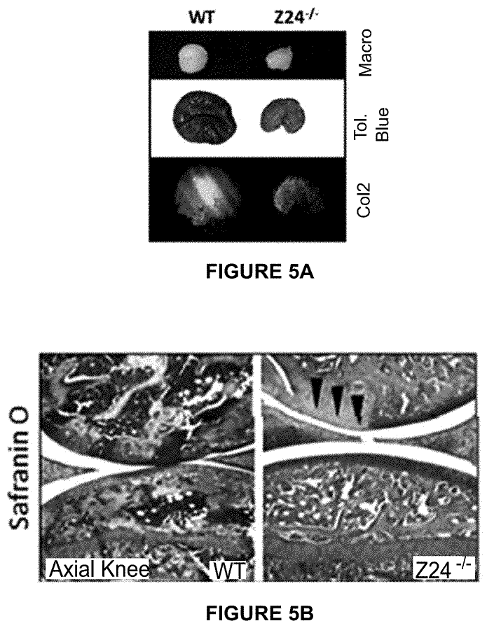

Z24−/− mice are deficient in the metalloprotease Zmpste24, which results in the accumulation of unprocessed Lamin A, very similar to progerin in HGPS patients, causing blebbing of the nuclear membrane and leading to destabilization of heterochromatin, DNA damage, and eventual cell cycle arrest and senescence. Chondrocytes are the primary cell type in articular cartilage (AC) and are responsible for maintaining the specialized extracellular matrix proteoglycan on joint surfaces. To gauge the effects of Zmpste24 loss specifically in chondrocytes, primary costal chondrocytes from 2 month old Z24−/− mice were isolated as previously described (Brittberg, et al. 2001 Clin Orthop Relat Res 2001(391 Suppl):S337-48). Accordingly, pellet culture of isolated chondrocytes were significantly smaller, with decreased Col2 signal and chondrogenic capacity (per toluidine blue stain intensity) (FIG. 5A). Safranin O staining of Z24−/− AC revealed obvious loss of proteoglycan content versus WT at only 5 months of age (FIG. 5B). These data indicate that progeroid Z24−/− chondrocytes are sensitive to senescence and dysfunction, which may initiate or potentiate OA.

The Zmpste24−/− (Z24−/−) model of Hutchinson-Gilford Progeria Syndrome (HGPS) was used to model not only HGPS musculoskeletal decline, but also as a pre-clinical aging model due to its predictable and accelerated aging phenotypes. Z24−/− animals are short lived (˜6 months) and have severe musculoskeletal abnormalities including weight loss, dystonia, sarcopenia, osteoporosis and as reported herein, early signs of bone loss and spontaneous osteoarthritis (OA). The data in this animal model suggest that progeroid Z24−/− chondrocytes are sensitive to senescence, which may initiate or potentiate OA. Indeed, safranin 0 staining of Z24−/− articular cartilage revealed obvious loss of proteoglycan content versus WT at only 5 months of age.

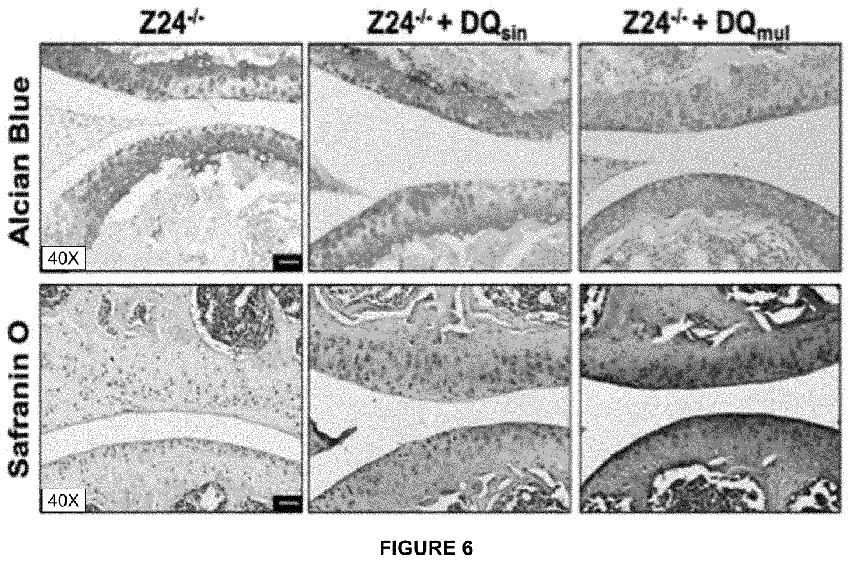

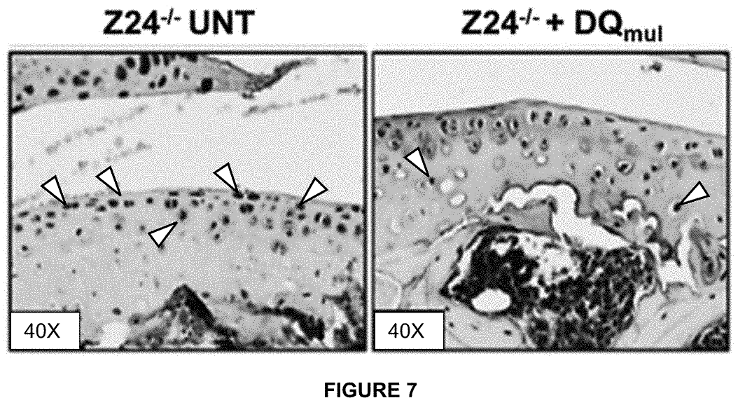

As a proof of concept for the potential of senolytics to reduce AC degeneration, a pilot study was conducted with Dasatinib+Quercitin (D/Q; 5 mg/kg; 50 mg/kg). Z24−/− mice were administered D/Q via oral gavage as a single dose (D/Qₛᵢₙ) at 4 months of age, or monthly dose starting at 2 months of age (D/Qₘᵤₗ) prior to sacrifice at 5 months. A single dose of D/Q was sufficient to mitigate proteoglycan loss in AC of Z24−/− mice (FIG. 6). Mice treated with multiple doses of D/Q demonstrated an even greater, dose-dependent response. In addition, Z24−/− mice in the D/Qₘᵤₗ group were found to have decreased ADMTS-4 positive cells, indicative of less ECM degradation (FIG. 7). While only preliminary, and from a single time point (5 months), these data strongly indicate that senolytic drug treatment may facilitate the retention of AC proteoglycan content and mitigate age-related OA pathogenesis.

Thus, it was also demonstrated that treatment with senolytic drugs (including Fisetin, Dasatinib, Quercetin, and Alvespimycin) can reduce the incidence/severity of many age-related disorders including osteoarthritis (OA) in this progeroid mice accelerated aging model. Senolytic treatment (D/Q and Fisetin) was shown to delay articular cartilage degeneration in a murine model of natural and accelerated (progeria) aging. Based on this promising pre-clinical data, a phase-1/2 clinical trial was proposed to investigate the efficacy of senolytic agents to reduce senescent cells and SASP factors in BMSC and consequently improve their beneficial effect for OA patients.

### Example 2. Blocking TGF β1 with Losartan Improves BMSC Therapy for Treatment of Knee OA

Blocking TGF-β1 with Oral Losartan Administration Improves AC Healing in a Rabbit Osteochondral Defect Model.

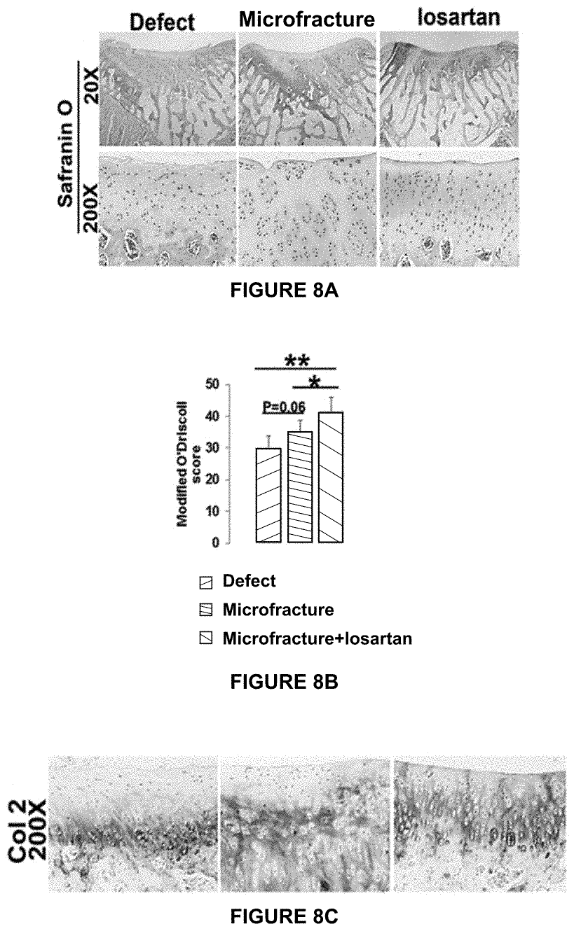

An osteochondral defect (diameter: 5 mm, depth: 2 mm) was made in the patellar groove of 48 New Zealand White rabbits. The rabbits were divided into 3 groups (8/group/time point): control (osteochondral defect only), microfracture (defect+microfracture), and Losartan (defect+microfracture+Losartan). For the Losartan group, a dose of 10 mg/kg/day of Losartan was orally administrated by mixing with the chew treat daily. At 6 weeks after surgery, the Losartan group showed significantly improved articular cartilage repair compared to microfracture or control groups (Utsunomiya, et al. 2019 Orthopaedic J Sports Med 7(7_supp15):2325967119S00263). At 12 weeks, the ICRS macroscopic score of the Losartan group (11.7±0.5) was significantly higher than control group (8.3±2.1, P<0.001) and microfracture group (7.5±1.2, P=0.003). The modified O'Driscoll score (based on Safranin O staining) was significantly higher with Losartan compared to the other 2 groups (FIGS. 8A and 8B). Immunohistochemistry staining of cartilage in the Losartan treatment group showed nearly normal structure with hyaline-like column-arranged chondrocytes (FIG. 8C). These results indicate that blocking TGF-β1 with Losartan can improve AC repair through prevention of fibrocartilage.

### Anti-Fibrotic Agents can Improve the Regenerative Potential of Adult Stem Cells by Preventing Fibrosis.

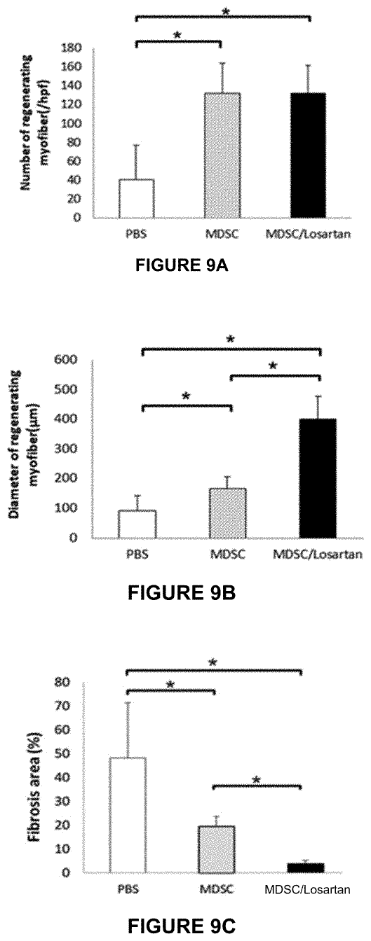

Adult muscle-derived stem cells (MDSC) can undergo myofibroblast differentiation and contribute to fibrosis under the influence of TGF-β1 released in the injured muscle. A study was performed to determine whether the transplantation of MDSCs in the presence of the TGF-β1 antagonist Losartan would result in decreased scar tissue formation and consequently enhanced muscle regeneration after injury in a mouse model. Compared to MDSC transplantation alone, MDSC plus Losartan treatment resulted in significantly decreased scar formation, an increase in the number of regenerating myofibers (FIGS. 9A-9C) and greater muscle force generation (Kobayashi, et al. 2016 Am J Sports Med 44(12):3252-3261). These experiments support that blocking fibrosis with Losartan can improve the regenerative potential of stem cells through prevention of fibrosis. Per the instant disclosure, Losartan may also improve the regenerative potential of BSMCs for AC repair after OA.

### Example 3. Cartilage Healing Following Bone Marrow Stimulation (Microfracture) in Conjunction with Fisetin, Losartan and Bone Marrow Aspirate Concentrate in a Rabbit Osteochondral Defect Model

Microfracture or Bone marrow stimulation (BMS) is the most commonly used first-line treatment for cartilage injuries; however, it has been shown to have inferior long-term clinical outcomes, primarily due to the production of fibrocartilage (not pure hyaline cartilage), i.e., the development of fibrosis in the repair tissue. Transforming growth factor (TGF-β1) can promote fibrosis, but it can be inhibited by the administration of Losartan to significantly reduce fibrocartilage. Losartan administration was found to enhance microfracture for cartilage repair (Utsunomiya, et al. 2020 AJSM 48(4):974-984). Furthermore, as mentioned in Example 2, Losartan administration can enhance BMS for cartilage repair. Indeed, oral administration of Losartan was shown to result in improved cartilage repair after BMS and to increase hyaline-like cartilage tissue. However, Losartan has the side effect of hypotension.

Fisetin, a senolytic flavonoid primarily found in strawberries that has been shown to extend the health and lifespan in anti-aging studies, has likewise been shown to decrease cartilage destruction and subchondral bone plate thickness in mice osteoarthritis (OA) models. However, the effect of Fisetin is yet to be investigated in osteochondral defect (OD) models.

The potentially synergistic effect of adding BMAC and Losartan to Fisetin has not yet been investigated in osteochondral models. The purpose of this example was to determine the effect of Fisetin, as well as its synergistic effects with Losartan and BMAC, for cartilage repair in a rabbit model.

Bone Marrow Aspirate Concentrate (BMAC) is another biological product that has demonstrated positive effects in bone and cartilage applications, including osteochondral defect, osteoarthritis and bone nonunion. BMAC has been well characterized as a source of mesenchymal stem cells (MSCs) and growth factors with regenerative capacity.

Thus, the combination therapy of the biologics with BMS was evaluated for the optimal and durable repair of cartilage defects. If the combined effect of Fisetin and Losartan is confirmed, the Losartan dose could possibly be lowered to avoid potential side effects. The supplementary effect on cartilage regeneration by co-administration of BMAC in surgical intervention was likewise examined.

### Study Design and Methodology

Bone Marrow Collection and BMAC Processing for Autologous Transplant: New Zealand White rabbits were anesthetized to collect bone marrow aspirate (BMA) using an 18 G heparinized spinal needle through both iliac crests simultaneously by two surgeons for the purpose of preparing bone marrow aspirate concentrate (BMAC). Bone marrow samples were collected in 10 ml syringes containing ACD-A as an anticoagulant (˜1.5 ml of ACD-A per 10 ml sample), processed under sterile BSCs, and a benchtop centrifuge was used to prepare BMACs. Filters were also used for BMA prior to processing into BMACs. These procedures were performed under sterile conditions.

### Osteochondral Defect (OD) Creation and BMS Procedure

A 5 mm diameter osteochondral defect was created in the patellar groove of bilateral knees for 64 New Zealand White rabbits (128 knees). Under a sterile technique, a midline 3 cm incision was made in a knee flexed at approximately 30° and approached intra-articularly through a medial parapatellar incision. The patella was dislocated, and a 5 mm diameter osteochondral defect was created in the patellar groove (depth: 2 mm), followed by BMS. To allow bone marrow MSCs to be introduced into the defect space, five equally spaced holes of 2 mm depth were drilled into the subchondral bone using a 0.7 mm burr. Blood from the bone marrow was observed to ooze into the cartilage defect through each BMS hole. Once the creation of the osteochondral defect and the BMS procedure was completed, the joint capsule was closed and 0.5 ml of BMAC was injected into the joint cavity of the left knee.

Post-surgical evaluations were to be conducted at two time points: 6 & 12 weeks. Autologous BMAC was injected into the unilateral knee joint cavity immediately after surgery, with no BMAC injection on the other side. Each rabbit was orally treated with Fisetin, Losartan, or a combination of the two biologics from the day after surgery until the day of euthanasia, for comparison with the untreated group. Rabbits were sacrificed 6 weeks or 12 weeks postoperatively. The experimental groups were as follows (N-8 per group): 1) OD+BMS; 2) OD+BMS+oral Fisetin; 3) OD+BMS+oral Losartan; 4) OD+BMS+oral Fisetin+oral Losartan; 5) OD+BMS+BMAC; 6) OD+BMS+BMAC+oral Fisetin; 7) OD+BMS+BMAC+oral Losartan; and 8) OD+BMS+oral Fisetin+oral Losartan.

### Preliminary Results

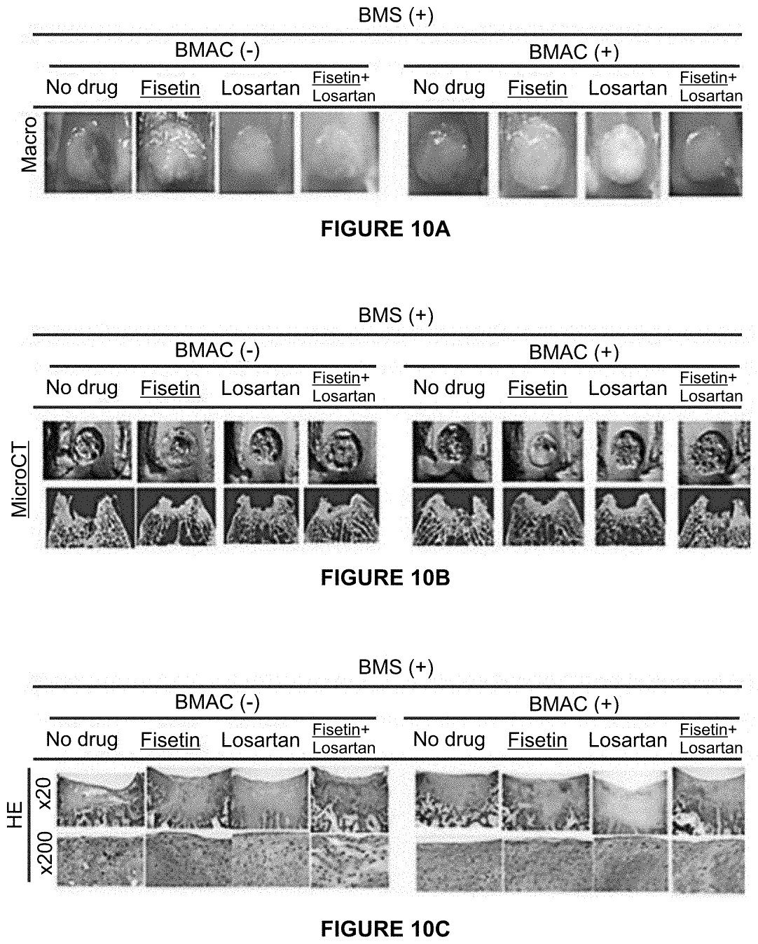

Macroscopic assessment and microCT: at 6 weeks postoperatively, gross images showed a significant improvement in the Fisetin group, BMAC group, and BMAC+Fisetin group compared to the control group (with BMS, without medication, and without BMAC). Micro CT examination showed healing of the subchondral bone in each treatment group, compared with limited healing in the control group (FIGS. 10A-10C).

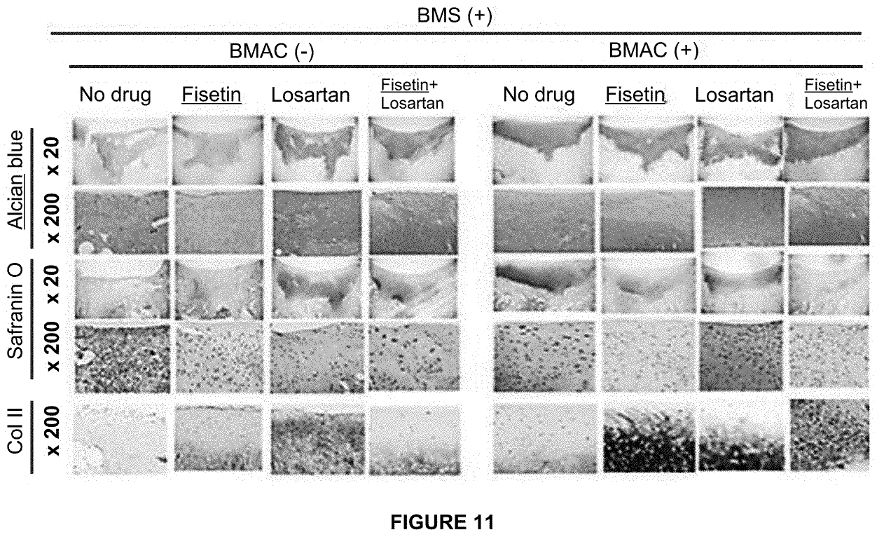

Histology: H&E staining showed superior osteochondral defect healing with cartilage tissue in each treatment group compared to the control group, which showed obvious fatty infiltration and fibrosis. Alcian blue and SO staining showed intense blue or orange-red cartilage matrix formation in each treatment group. Chondrocytes showed an organized hyaline like morphology in each treatment group compared to fibrocartilage in the control group. Furthermore, immunohistochemistry showed stronger collagen II staining (brown color) in the superficial and proliferative areas of the regenerated cartilage in each treatment group than in the control group (FIG. 11).

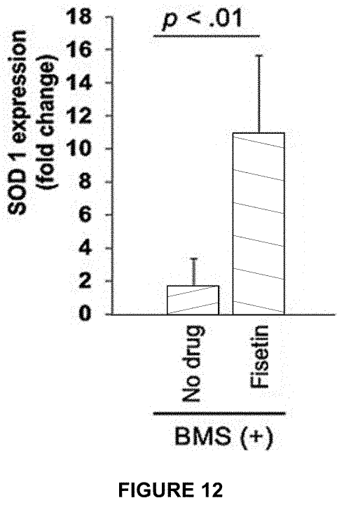

Gene expression analysis: qPCR showed that the expression level of Superoxide Dismutase 1 (SOD1) in the synovium harvested from the knee joint was significantly higher in the Fisetin-treated group than in the control group (p<0.01), indicating that the antioxidant effect of Fisetin was enhanced (FIG. 12).

### Preliminary Results at 12 Weeks Postoperatively

Microscopic evaluation and micro-CT: results of preliminary experiments at 12 weeks postoperatively showed a trend toward improvement with Fisetin, Losartan, or a combination of the two biologics, with or without BMAC. Only the Fisetin-Losartan group showed a significant increase in cartilage repair compared to the control group. However, due to the small number of samples in each group in the pilot studies, additional samples were added for further analysis.

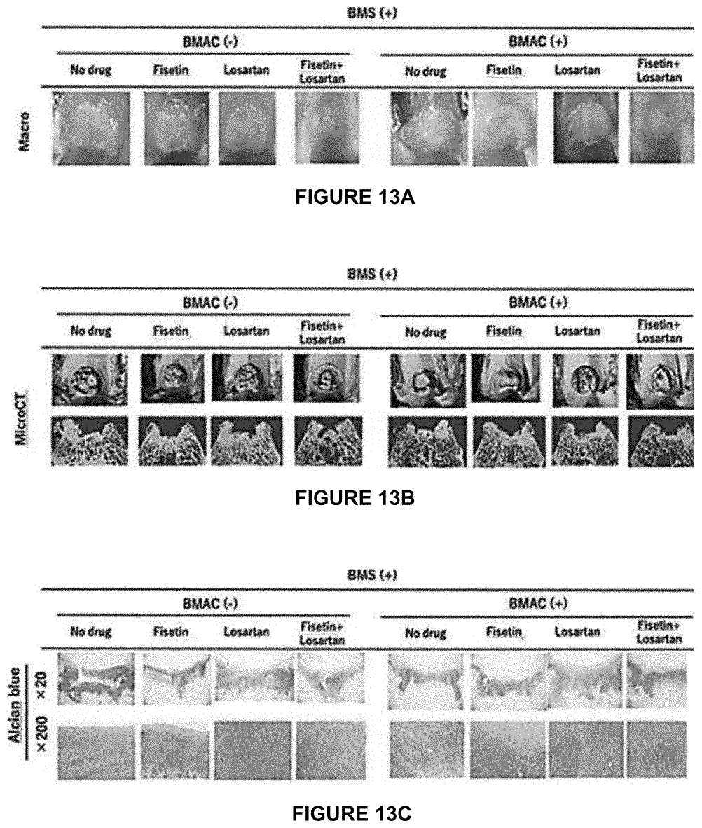

Histology: H&E staining showed excellent healing of the osteochondral defect by cartilage tissue in each treatment group, whereas there was obvious fatty infiltration and fibrosis in the control group. Alcian blue and SO staining showed strong blue or orange-red cartilage matrix formation in each treatment group. Alcian blue staining at 12 weeks showed evenly distributed blue matrices (hyaluronic acid, acid sulfate) compared to 6 weeks cartilage in all groups. Chondrocytes showed an organized hyaline morphology in each treatment group compared to fibrocartilage in the control group (FIGS. 13A-13C).

The preliminary results showed that Fisetin, Losartan, and combinations of these biologics enhanced the healing capacity of BMS-mediated osteochondral defects compared with BMS alone. Furthermore, BMAC administration may further enhance these benefits. Oxidative stress is one of the inducers of cell senescence, which has been noted as the primary factor contributing to age-related changes in cartilage homeostasis, function, and response to injury. The results revealed that SOD1 was significantly higher in the synovium of Fisetin-treated rabbits, suggesting that the antioxidant effect of Fisetin could have contributed to the improvement of cartilage healing observed.

BMAC intra-articular injection to enhance the BMS procedure for the treatment of osteochondral defects is also contemplated.

### Example 4. Study of Use of Fisetin and Losartan to Improve the Beneficial Effect of Bone Marrow Stem Cells for the Treatment of Osteoarthritis

As noted above, in vitro expansion of adult stem cells leads to senescence. Senescent cells can induce an osteoarthritis-like condition in mice. A number of compounds that selectively target and kill senescent cells have been identified and characterized as novel senolytic drugs (Kirkland and Tchkonia 2017 EBioMedicine 21:21-28). These senolytic drugs target and inhibit anti-apoptotic pathways that are upregulated in senescent cells, thereby inducing apoptotic cell death and abrogating systemic SASP factors. Using senolytic drug-containing medium to expand stem cells is shown to eliminate senescent cells during adult stem cell expansion without altering stem cell function. Furthermore, the number of senescent cells as well as SASPs factors increased in BMSCs with age. Thus, eliminating senescent cells and SASP in BMSCs with senolytic treatment should improve the ability of BMSCs to promote cartilage repair after OA. Blocking fibrosis is shown to improve the regenerative potential of adult stem cells. Additionally, the use of Losartan alone can improve hyaline cartilage repair while reducing the amount of fibrocartilage. Thus, it was posited that reducing fibrosis with Losartan would improve the beneficial effect of BMSCs on AC repair after OA, when compared to BMSCs alone. It was also posited that a synergistic beneficial effect would be observed by combining senolytic agents with Losartan to eliminate senescent cells and reduce fibrosis, which would consequently contribute to an optimal effect of BMSCs for OA patients when compared to either treatments used individually.

A randomized, double-blind, placebo-controlled clinical trial was designed to evaluate: i) the ability of Fisetin (FIS), a widely available dietary supplement, to improve the clinical efficacy of bone marrow stem cells for the treatment of knee osteoarthritis; ii) the ability of Losartan (LOS), an FDA-approved drug with an established safety profile, to improve the clinical efficacy of bone marrow stem cells for the treatment of knee osteoarthritis; and iii) the ability of combined FIS and LOS to have a greater, synergistic effect on the clinical efficacy of BMSCs for the treatment of knee OA when compared to either treatments (LOS and FIS) used individually (Table 1, below). Certain patient populations and assessments were built on NCT04210986.

TABLE 1

| BMC for Knee | | Losartan | |
|---|---|---|---|
| OA | | No | Yes |
| Fisetin | No | BMSC Only | BMSC + Losartan |
| | | (placebo fisetin and | (with placebo fisetin) |
| | | placebo Losartan) | |
| | Yes | BMSC + Fisetin | BMSC + Losartan + Fisetin |
| | | (with placebo Losartan) | |

Data was also collected to determine the extent to which FIS treatment reduces senescent cells and pro-inflammatory and degenerative cartilage SASP markers in BMA, BMSCs, and peripheral blood

### Proposed Target Subject Population and Indication

The targeted subject population includes subjects with symptomatic knee OA with Kellgren-Lawrence grade II-IV. A summary of the key inclusion/exclusion criteria is provided below.

Inclusion Criteria are: 1) Capacity to give informed consent and willing to comply with all study related procedures and assessments. 2)≥40 and ≤85 years of age. 3) Ambulatory persons with unilateral or bilateral osteoarthritis (OA) of the knee and baseline pain with a mean of ≥3 and ≤9 points on the 24-hour mean pain score (on the 11-point Numeric Rating Scale) for at least five of the seven days during the screening period.

Exclusion Criteria are: 1) Any condition, including laboratory findings and findings in the medical history or in the pre-study assessments, that constitute a risk or contraindication for participation in the study or that could interfere with the study objectives, conduct, or evaluation or prevent the subject from fully participating in all aspects of the study, 2) Clinically significant co-existing conditions that significantly compromise overall health, 3) Patients with a history of diabetes mellitus, 4) History of cardiac rhythm disturbances, abnormal ECG intervals, or use of medications known to impact ECG intervals, 5) Previous or planned surgery on the target knee (other than arthroscopy for diagnosis and/or debridement only), 6) Intra-articular treatment with steroids or hyaluronic acid derivatives within the last 12 weeks prior to screening, 7) Regenerative joint procedures on any joint including, but not limited to, platelet-rich plasma injections, mesenchymal stem cell transplantation, autologous chondrocyte transplantation, or mosaicplasty within the past 6 months, 8) Current or prior history of other significant joint diseases, 9) Any active known or suspected systemic autoimmune disease or any history of systemic inflammatory arthritis, 10) Current diagnosis and symptoms of fibromyalgia, 11) BMI≥40 kg/m2, or size exceeding the limits of the of the MRI equipment (coil and gantry), 12) Patients with a history of active blood disorders or cancers, 13) Patients that are not able to have a minimum of 90 mL of BMA harvested due to collection and/or processing complications, 14) Patients already taking a senolytic, Losartan or closely related medications.

### Study Treatments

All subjects underwent a posterior-superior iliac spine bone marrow harvest procedure and intra-articular injection of BMC (containing BMSCs) in the knee joint. The Fisetin+placebo, Losartan+placebo, and Fisetin+Losartan treatment groups received two bottles, one containing 100 mg and the other 25 mg capsules, to be administered orally. The Fisetin (and the corresponding placebo) used is purchased from a contracted GMP manufacturer. Fisetin capsules are size #3 and opaque blue in color. The placebo comparator is mainly composed of cellulose along with some coloring agent and is manufactured using the same size and color capsule to mimic the appearance of the active Fisetin capsules. The Losartan used is manufactured under cGMP conditions, supplied in 25 mg capsules. Oral Losartan potassium and appearance-matched placebo capsules are produced by a compounding pharmacy. In the instant study of non-hypertensive subjects, subjects received 25 mg daily taken orally, in two 12.5 mg doses of Losartan potassium capsules or matching placebo capsules for all treatment groups.

### Experimental Procedures

Pre-Procedure Visit (Baseline): After screening and obtaining informed consent, baseline EKG, objective, demographic, functional performance, imaging (MRI) data, and senescence profiling were done prior to the BMSC treatment. Group randomization occurred after baseline data is reviewed to confirm subject eligibility. Subjects failing any of the screening criteria were excluded from further study participation.

### Blood Draw (Baseline, 14 Days, 1 Month, 3 Months, 6 Months, 18 Months)

A sample of blood obtained via IV or venipuncture was used for general health and AE screening. For research purposes, approximately 30 mL of blood were drawn into labeled lavender and red top vacutainer tubes. Approximately 0.8 mL of whole blood sample from the lavender top tube was used to measure blood cells, a complete blood count (CBC) with differentials. The tubes were centrifuged to separate buffy coat and plasma. Following centrifugation, the buffy coat (containing T-Cells) was isolated and analyzed for senescence. The plasma was aliquoted into microcentrifuge tubes with the subject's research ID number and stored at −80° C. until batch multiplex immunoassay and analysis was performed.

### Bone Marrow Harvest Procedure

Bone marrow harvest procedures were performed in a clinical setting and patients monitored throughout with a pulse oximeter, oxygen tank, nasal cannula (to supply oxygen), and/or blood pressure cuff. BMA was harvested using the same collection technique as previously described (Chahla, et al. 2017 Arthrosc Tech 6(2):e441-e445). Briefly, the subject was placed in the prone position, and the harvest site was sterilely prepped and draped. The bony landmarks of the posterior superior iliac spine (PSIS) were located by palpation and confirmed using ultrasound guidance. Local anesthetics were administered into the superficial layers of the skin. A BMA kit (Arrow OnControl, Teleflex, Shavano Park, Tex.) was opened and a battery-powered aspiration drill was sterilely draped. Then, an 11-gauge ported aspiration needle was percutaneously inserted through the skin and subcutaneous tissues until reaching the PSIS. The battery-powered intra-osseous drill (Arrow OnControl, Teleflex, Shavano Park, Tex.) was then used to insert the ported aspiration needle into the medullary cavity of the PSIS. A syringe preloaded with 1 mL of anticoagulant citrate dextrose solution formula-A (ACD-A) was injected into the site to minimize coagulation. Up to 120 mL of BMA were collected unilaterally form the PSIS in 30 mL syringes preloaded with 5 mL of ACD-A. The harvest steps were repeated on the contralateral side of the PSIS, if necessary, to obtain a sufficient volume of aspirate. BMA samples were immediately labeled and cross-referenced with the patient's ID # and CBC form. The BMA was then taken to a separate clinical laboratory for processing. Up to 30 ml BMA were used for research purposes to evaluate the senescence profile. Following BMA centrifugation, the buffy coat (containing T-Cells) was isolated and analyzed for senescence and various cellular signatures (MSCs, HSCs, pericytes, etc.) using a protocol previously described (Crisan, et al. 2008 Cell Stem Cell 3(3):301-13; Zheng, et al. 2007 Nat Biotechnol 25(9):1025-34). The plasma was aliquoted into microcentrifuge tubes with the subject's research ID number and stored at −80° C. until batch multiplex immunoassay and analysis was performed.

### Bone Marrow Processing Technique

Under a biosafety hood, 1 mL of BMA was extracted and transferred to measure cellular concentrations via flow cytometry and 0.8 mL of BMA for a complete blood count. The microcentrifuge tubes were labeled with the subject's ID number and placed in a biohazard bag. The sample was processed to completion. On the last step, 0.8 mL of bone marrow concentrate (BMC) was extracted and transferred to a microcentrifuge tube (labeled with the subject's ID number) to measure blood cell quantities using the CellDyn Ruby hematology analyzer.

### Knee Injection Procedure

The BMSC sample was cross-referenced with the treating clinician. An injection tray was opened in a sterile fashion, and the injectate solution was poured into a sterile medicine cup. The subject was placed in a supine position, and the knee was prepared and draped in a sterile fashion. After local anesthetics were administered into the superficial tissues of the knee, an 18- or 22-gauge, 3.5-inch needle was advanced using a lateral suprapatellar approach. Finally, 6-9 mL of BMSC was injected into the intra-articular space of the knee.

### Synovial Fluid Collection (Baseline, 6 Months, 18 Months)

Synovial fluid (1-10 mL) was collected on the day of procedure, and a separate arthrocentesis procedure was performed at 6-month and 18-month time points. Once the skin was anesthetized, synovial fluid was collected into sterile syringes using a lateral suprapatellar approach with an 18- or 22-gauge needle, and then immediately transferred to a sterile centrifuge tube under a biosafety hood. After proper balancing, the sample was centrifuged for 15 minutes at 3500 RPM. After centrifugation, the supernatant was removed, taking care not to disturb the cellular pellet. The top fraction of the synovial fluid was extracted and transferred to cryovials. All samples were properly labeled with corresponding subject ID number and frozen at −80° C. for batch multiplex immunoassay and analysis to measure SASP and OA biomarkers.

### BMC/BMSC Characterization

Flow Cytometry Analysis: To confirm stem cell populations, cells were stained with the following antibodies: CD31-V450 (1:400) or CD144-PerCP Cy5.5 (1:100), CD34-PE (1:100), CD45-V450 (1:400) or CD45-APC Cy7 (1:100), and CD146-BV711 (1:100) (BD Biosciences, San Jose, Calif.). Cells were stained for 30 minutes at 4° C., followed by washing with 2% FCS/PBS. Analysis was performed on a flow cytometer (Miltenyi Biotec). Data is analyzed using the Miltenyi Biotec platform software, and the percentage of Mesenchymal stem cells (CD73+, CD90+, CD105+), Hematopoietic stem cells (CD45+, CD34+, CD59+, CD90+), Endothelial cells (CD31+, CD105+), and Pericytes (CD146+, CD34−, CD45−) is determined for each preparation of BMSCs.

Patient-Reported Outcomes (baseline, 1 month, 3 months, 6 months, 12 months, 18 months): Self-reported physical function was assessed by the Western Ontario McMaster Osteoarthritis Index (WOMAC) and was used to evaluate knee-specific impairments. The WOMAC holds three separately scored subscales, including pain, function and stiffness. The WOMAC has been validated for OA, total joint arthroplasty, and rehabilitation outcomes (Angst, et al. 2005 J Rheumatol 32(7):1324-30). The effect size is generally largest for the subscale QOL (Quality of Life) followed by the subscale Pain. In addition to WOMAC, IKDC and SF-12 forms were also collected; all three are valid, reliable, and responsive self-administered questionnaires that can be used for short-term and long-term follow-up of knee injury, including osteoarthritis (Roos and Toksvig-Larsen 2003 Health Qual Life Outcomes 1(1):17; Roos, et al. 1998 J Orthop Sports Phys Ther 28(2):88-96).

Performance-Based Physical Function (baseline, 6 months, 18 months): Measures of functional performance include 6 min walk test (6MW), timed-up-and-go test (TUG), and 4-meter walk (4 mW) tests (Moffet, et al. 2004 Arch Phys Med Rehabil 85(4):546-56; Parent and Moffet 2002 Arch Phys Med Rehabil 83(1):70-80; Parent and Moffet 2003 Arthritis Rheum 49(1):36-50). The 6MW test measures the distance walked in 6 minutes to measure endurance. It is safe, easy to administer, well tolerated, and has excellent test-retest reliability (ICC 0.95-0.97) and a low coefficient of variation (10.4%). The timed up and go (TUG) measures the time it takes a patient to rise from an arm chair (seat height of 46 cm), walk 3 m, turn, and return to sitting in the same chair without physical assistance, and also has excellent reliability. The 4 mW test assesses the capacity for performance of certain activities (e.g. crossing a street before the light changes) and is assessed at the fastest safe speed for each participant. The 4 mW test was selected, because: 1) it has been shown to predict risk of mobility and physical disability, higher health care utilization, and increased mortality; 2) it is a meaningful outcome measure in older persons with a wide range of conditions; 3) it is valid and reliable; and 4) it is well tolerated by patients varying in condition and degree of health. The time it takes for each participant to ascend and descend 9-12 stairs is measured to assess joint strength, stability, and agility.

### Biomarker Assessment for Senescence and OA

Biomarker assessment for senescence and OA was performed using plasma obtained from whole blood at baseline, 14 days, 1 month, 3 months, 6 months, 18 months, and BMA. OA-related biomarkers to be evaluated include: matrix metalloproteinases (MMPs), interleukins, adipokines and joint related serum biomarkers MMP-degraded C-reactive protein (CRPM), MMP degraded type III collagen (C3M), cartilage oligomeric matrix protein (COMP), HA, N-terminal propeptide of collagen IIA (PIIANP), Col2-3/4 C-terminal cleavage product of types I and II collagen, uCTX-II, matrix metalloproteinase-3 (MMP-3) and urinary nitrated type II collagen degradation fragment (uCol2-1 NO₂) (Watt 2018 Osteoarthritis Cartilage 26(3):312-318; Mobasheri, et al. 2017 Osteoarthritis Cartilage 25(2):199-208). Other newer biomarkers believed to be associated with OA can also be tested in blood and/or synovial fluid samples from knee OA patients such as: sHA (rho=0.19), PIIANP (rho=0.27) and C1, 2C (rho=0.20), uCTX-II, MMP-3, uCol2-1 NO₂ and sHA. Cartilage damage and concentrations of sCOMP, sCTX-II, sMMP-3, sPIIINP, and sHA can also be tested (Jiao, et al. 2016 Biomarkers 21(2):146-51). PIIANP, serum CTX-II, HA, and COMP can also be measured, because they were found to be significantly higher in the knee OA patients with early signs of cartilage damage. Plasma levels of pro-inflammatory senescence associated secretory phenotype (SASP) factors GM-CSF, IL-1β, IL-6, IL-8, IL-10, IFNγ, and TNF-α, were measured via a commercially available multiplex assay following manufacturer's instructions (meso scale delivery, K15007B-1). Of note, these factors are not only senescence-associated factors, but are strongly associated with OA, thus allowing the simultaneous detection of both senescence and OA biomarkers. In addition, stress markers associated with aging were measured including DNA damage markers CRAMP, EF-1α [100] and oxidative/nitrosative stress markers 4-hydroxynonenal (4-HNE), malondialdehyde (MDA), and 3-nitro-tyrosine β-NT) via commercially available immunoassays as described (Marrocco and Peluso 2017 Oxid Med Cell Longev 6501046). For all assays using plasma, analysis was performed under masked conditions using serum or EDTA-treated plasma. Detection of these factors in coordination with distinct OA biomarkers described above help determine the efficacy of senolytic and anti-fibrotic drugs for reducing mediators directly related to OA-related cartilage degeneration, inflammation, and pain.

### Magnetic Resonance Imaging (Baseline, 6 Months, 18 Months)

Quantitative 3-Tesla magnetic resonance imaging (MRI) was utilized to assess articular cartilage (AC) quality: 1) MRI relaxometry including T2 mapping of the AC in the knee; 2) MRI-based 3D volumetry/shape quantification to allow measurement of cartilage thickness and joint morphology.

### Inclusion of MR Imaging Data

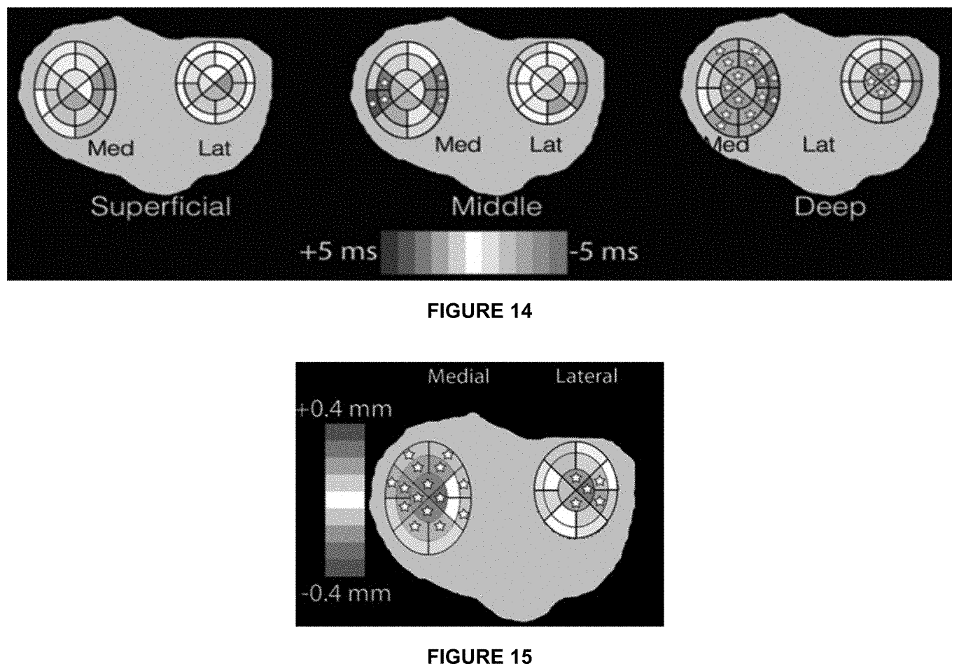

T2 Mapping: T2 mapping is sensitive to collagen matrix structure and water content of the cartilage, with significant differences between intact and damaged cartilage as validated with arthroscopic and histological measurements (Ho, et al. 2016 Arthroscopy 32(8):1601-11). Cartilage T2 mapping values has also been shown to change significantly between different stages of OA, as well as between different surgical repair techniques and various pathologies (Ferro, et al. 2015 Arthroscopy 31(8):1497-506; Russell, et al. 2016 J Orthopaedic Res: Official Pub of the Orthopaedic Res Soc.; Oneto, et al. 2010 Knee Surg Sports Traumatol Arthrosc 18(11):1545-50). T2 relaxation time measures were correlated with patient-reported outcomes 6 and 18 months after BMSC treatment (Su, et al. 2016 Osteoarthritis Cartilage 24(7):1180-9). T2* relaxation time imaging has also been used to evaluate cartilage quality after attempted cartilage repair using autologous chondrocyte transplantation (Welsch, et al. 2010 Eur Radiol 20(6):1515-23) and microfracture (Oneto, et al. 2010 Knee Surg Sports Traumatol Arthrosc 18(11):1545-50). This technique is sufficiently sensitive for identifying changes in cartilage properties over relatively short periods of time. Depth-dependent significant differences in cartilage T2* on the medial tibial plateau between ACL-injured and contralateral knees as early as six months after ACL injury/reconstruction that progressed from 6 to 24 months (FIG. 14) have also been observed. Relaxation times for deep layer tibial cartilage were consistently shorter than in uninjured knees, suggesting thickening of the calcified cartilage layer, which may be a predecessor to osteoarthritic change. This clearly demonstrates the sensitivity of MRI T2* mapping to osteoarthritic cartilage changes.

### 3D Volumetry/Shape Quantification

High-resolution, near-isotropic sequences have previously been used to characterize 3D changes in cartilage morphology/thickness. MRI scanning (Siemens 3T Magnetom Trio, near-isotropic 3D Dual Echo Steady State (DESS) with water excitation, CP Extremity knee coil, voxel size: 0.45×0.45×0.70 mm, TR: 16.32 ms, TE: 4.71 ms, Flip Angle=25°, 140×140 mm field of view) was performed bilaterally on 50 subjects 6 and 24 months after ACL reconstruction. Cartilage was segmented manually using Mimics software (Materialize, Belgium), and mapped to regions defined based on anatomical landmarks (Wirth and Eckstein 2008 IEEE Trans Med Imaging 27(6):737-44; Anderst, et al. 2008 A Technique for Calculating and Mapping Focal Cartilage Thickness, in North American Congress on Biomechanics (NACOB)). Accuracy of the resulting 3D cartilage maps was verified by cadaver study, comparing MRI-measured cartilage thickness with high-accuracy 3D laser scans, with average thickness errors of 0.09±0.27 mm for femurs and 0.05±0.19 mm for tibias (Thorhauer and Tashman 2015 Med Eng Phys 37(10):937-47). Cartilage thickness was unchanged in the uninjured knees, but was increased significantly in the ACL injured/reconstructed knees from 6 to 24 months after reconstruction surgery (FIG. 15). Cartilage hypertrophy was observed during the early time period after joint injury has been previously reported (Eckstein, et al. 2015 Arthritis Rheumatol 67(1):152-61) and may be a sign of early arthritic changes in human knees (Cotofana, et al. 2012 Arthritis Care & Research 64(11):1681-1690). This tool has demonstrated sensitivity to address relatively short-term changes in cartilage morphology.

### Magnetic Resonance Imaging Protocol

MRI scanning was performed on the 3 Tesla clinical scanner (Skyrafit 3T, Siemens Medical Solutions). Optimized protocols based on prior work and a knee-specific coil were used. Scans were acquired for the affected knee of each subject at baseline and repeated 6 months and 18 months after BMC injection. Each series of scans required one hour or less to complete.

### MRI Relaxometry

T2 map imaging was acquired using a 7-echo sequence (echo times 13.8-96.6 ms; resolution 0.5×0.5×2 mm). Relaxometry map images were manually segmented to select tissue-specific regions of interest using Materialise Mimics (Materialise, Plymouth, Mich.) and a tablet monitor and stylus, as previously described (Surowiec, et al. 2014 Knee Surg Sports Traumatol Arthrosc 22(6):1385-95). Anatomical landmarks were identified and marked in Mimics to allow landmark-based division of each region of interest into clinically relevant anatomical sub-regions. MRI relaxometry values were extracted from each sub-region using custom MATLAB software (MATLAB, Mathworks, Natick, Mass.) and then analyzed. T2 map values from superficial, central, and deep layers were analyzed both combined and separately to accommodate for depth-specific changes (Wirth, et al. 2016 Sci Rep 6:34202).

### MRI-Based 3D Volumetry/Shape Quantification

Thin-slice, high resolution volumetric MR images (PDw FS SPACE; 0.6×0.6×0.7 mm resolution) were acquired to facilitate measurement of cartilage thickness and changes in joint morphology. The collected 3D acquisition images were manually segmented to create 3D cartilage models using Materialize Mimics (Materialize, Plymouth, Mich.) and a tablet monitor and stylus using the previously mentioned, validated manual segmentation techniques. Though the primary analyses are based on anatomic sub-region values, Statistical Parametric Mapping (SPM) techniques (Gallo, et al. 2016 Osteoarthritis Cartilage 24(8):1399-407) were used for secondary analyses to identify localized changes in T2 map values and cartilage thickness over time and compare them between groups. Standard clinical morphological images from a musculoskeletal/sports-medicine-optimized protocol were acquired for all subjects at 3T and evaluated. Semi-quantitative data were collected using structured joint-specific MRI finding data collection forms.

### Assessment of Biomechanical Joint Function (Baseline, 6 Months, 18 Months)

While MRI can identify structural and morphological joint changes, it cannot reveal joint function. Preservation of joint function and loading is a key factor for long-term joint health; previous studies have shown significant relationships between muscle strength and cartilage degeneration in individuals at high risk for OA (Macias-Hernandez, et al. 2016 Clin Rheumatol 35(8):2087-2092). A series of measurements to comprehensively assess changes at biomechanical function was carried out.

### Lower-Extremity Kinematics, Video Motion Analysis (Baseline, 6 Months, 18 Months)

Video-motion analysis can assess even subtle changes in musculoskeletal function due to limited joint range of motion, stiffness, pain, and/or weakness. Subjects were equipped with a full-body retro-reflective marker set (including four-marker thigh and shank clusters on each leg). Kinematics measurements were captured with a video-motion analysis system consisting of 18 infrared, 12 megapixel motion capture cameras (Oqus 7, Qualisys AB, Gothenburg, Sweden). Ground reaction forces were acquired simultaneously using an instrumented treadmill or force plates (Bertec, Columbus, Ohio). Angular kinematics and net joint moments (kinetics) were determined for the trunk, pelvis, hips, knees, and ankles using Visual3D software (C-Motion, Inc., Germantown, Md.). Tasks included treadmill gait (1 m/s) and stair ascent/descent. Primary outcomes were changes in joint range of motion and peak knee joint moments from baseline and following BMSC treatment.

### Assessment of Muscle Strength, Isokinetic Dynamometry (Baseline, 6 Months, 18 Months)

Muscle strength was assessed for both legs using a Humac Norm isokinetic testing system (Computer Sports Medicine Inc., Stoughton, Mass.). Subjects performed 3 repetitions of maximum-effort hip/knee flexion and extension at 60 degrees/s. Participants were evaluated in a seated position and performed a warm up with submaximal contractions prior to testing. All measurements were normalized to % body weight. Primary outcome is change in total work (TW) and peak torque (PT) from baseline to 18 months post treatment.

### Statistical Analysis Plan (SAP) Synopsis

A randomized 2×2 Factorial design was employed with a target minimum of 20 subjects per group with 18-month follow-up. 2-factor analysis of variance (ANOVA) is the primary statistical modeling method to assess differences in safety and efficacy attributable to fisetin, losartan, or a combination of fisetin and losartan, compared to BMSC treatment alone. To test for sex differences in treatment efficacy, and/or to control for baseline covariate, Analysis of Covariance (ANCOVA) method was used.

Additionally, for hypotheses regarding repeated post-treatment assessments, linear mixed effects modeling with random intercepts for each subject were used to test for losartan and/or fisetin group effects while adjusting for baseline value. In all cases of factorial analysis, the interaction term between fisetin and losartan administration was tested to determine if there was significant interference or potentiation associated with joint administration. If no statistically significant interaction term was found, this term was eliminated from the model, and main effects comparing 2n vs 2n subjects were reported (where n represents the per treatment group sample size). A statistical power calculation was performed based on previous studies (MacKay, et al. 2018 Osteoarthritis Cartilage 26(9):1140-1152), and 100 total patients were enrolled in a 1:1:1:1 allocation ratio (n=25 per treatment group).

The administration of senolytic drugs selectively eliminates senescent cells and senescent cell associated pro-inflammatory phenotypes (SASP factors) that are known to promote OA and should improve the beneficial effect of BMSCs for OA patients. OA symptoms should be delayed in both the Fisetin and Losartan treatment groups, when compared to the placebo group, as indicated by patient reported functional outcomes and differences in clinical chemistries. This includes reduction in plasma biomarkers for OA and senescence associated factors.

Minimal adverse events were anticipated at the outlined dosage and treatment regimen, as Fisetin is a naturally occurring flavonoids tolerable at high doses. However, if a significant number of severe adverse events were to emerge, Fisetin could be temporarily discontinued and/or dosage could be reduced. Given that the intermittent dosing of senolytic drugs is effective at eliminating senescent cells and SASP factors (2 days on and 28 days off), subtle modifications to dosage or treatment times were not expected to significantly alter predicted outcomes. Also, considering chondrocytes are in an avascular zone, beneficial effects conferred by senolytic agents would be indirect via a paracrine mechanism.

Minimal adverse events were anticipated at the outlined dosage and treatment regimen for Losartan, as well, as it is well tolerated with minimal side effects at the proposed dose. A synergistic beneficial effect was anticipated in combining senolytic agents with Losartan to eliminate senescent cells and reduce fibrosis, respectively, resulting in better outcomes of BMSC therapy for OA patients, when compared to either treatment used individually. Although the use of dietary supplements appears to be very common among patients who also take prescription medications, most potential drug—dietary supplement interactions have been found limited. Since the Fisetin treatment was 2 days on and 28 days off, OA patients were unlikely to take both drugs during the same day (when the Fisetin treatment is on 2 days per month, the Losartan medication could be eliminated, and for the remainder of the month, the OA patient could take only Losartan) in order to eliminate drug/dietary supplement interaction.

Finally, the predominant symptom associated with OA is pain, driven by inflammation, which leads to considerable physical and psychosocial disability (Hunter, et al. 2008 Rheum Dis Clin North Am 34(3):623-43). Reduction of inflammation due to senolytic drug treatment was expected to reduce pain, which might restore function to the joint. This was to be quantifiably assessed using biomotion analyses and isokinetic testing (at baseline, 6 months, and 18 months post-treatment). Any deviations from the proposed treatment regimens were addressed using an intent-to-treat approach.

When possible, samples from harvested bone marrow concentrate and peripheral blood (plasma and isolated buffy coat cells) were banked for future analysis. For clinical BMC treatment, it is common that not all of the BMC collected is used for injection.

### Example 5. Changes in Senescent Cells and SASP from Bone Marrow, Synovial Fluid, and Peripheral Blood after Senolytic Treatment

Cellular senescence is thought to be a fundamental driver of aging and major contributor to age-associated decline and loss of physiological reserve. Senescent cell accumulation is not only a fundamental property of aging, but also promotes several age-related morbidities such as osteoarthritis (OA), through the production of the Senescence Associated Secretory Phenotype (SASP). SASP factors include pro-inflammatory cytokines/chemokines, tissue degrading proteases, and reactive oxygen species (ROS) inducing signals responsible for paracrine induction and potentiation of inflammation and systemic senescence. Thus, understanding the spatiotemporal dynamics of senescent cell accumulation at the tissue and peripheral level, including cognate SASP production, is paramount to devise strategies to target senescence for the treatment of age associated chronic conditions.

Fisetin's demonstrated senolytic effects offer a potentially powerful and safe approach to promote healthy aging and delay age-related disease by selectively targeting and eliminating senescent cells without affecting quiescent or proliferating cells. Senolytic treatment (Fisetin) has been shown to delay articular cartilage degeneration in a murine model of OA. Thus, a phase-1/2 clinical trial is conducted to investigate the efficacy of senolytic agents to reduce senescent cells and SASP factors to consequently improve therapeutic approaches for OA patients. Senescent cells and SASP production are characterized locally and systemically in human synovial fluid, bone marrow, and peripheral blood collected as an added part of the trial described in Example 3, above. Samples for unique tissue compartments like synovial fluid and bone marrow can be difficult to obtain in healthy human patients.

The instant study is a randomized, double-blind, placebo-controlled clinical trial, in which samples of synovial fluid, bone marrow and peripheral blood mononuclear cells (PBMCs) are provided from 50 patients undergoing BMC injection with and without senolytic treatment. Clinical samples are collected and compared for senescent cells and SASP from three different compartments including peripheral blood, synovial fluid, and bone marrow. Changes in senescent cell content and senescence biomarkers are analyzed in these 3 compartments, with and without senolytic treatment with the dietary supplement Fisetin.

Senescence is associated with numerous phenotypic changes including cell cycle arrest that is accompanied by a distinct secretory profile. With the growing body of literature on this topic, it is important to highlight that senescence programs can differ among different cell types, and that senescence may exist in stages. The array of senescent phenotypes across space and time in the human body is thought to be programmed by cooperative metabolic and epigenomic changes that dictate the aging process. To gain a better understanding of the impact of senescent cells in individuals during the aging process, including the improvement of senotherapeutic strategies, a suite of technologies to detect, track, and interrogate senescent cells and SASP, in different tissue compartments, is paramount.

Senescent cells and SASP production locally and systemically in collected human synovial fluid, bone marrow, and peripheral blood as mentioned previously are characterized, with a view to identifying and analyzing senescent cells and associated SASP factors from synovial fluid, bone marrow, and peripheral blood, as well as to assessing the potential benefits of Fisetin for reducing senescence-related age-associated decline. Senescence characteristics in synovial fluid and bone marrow during aging are particularly underexplored, given that these tissues are incredibly common sites afflicted during age-related orthopedic decline such as OA and difficult to access. Clinical samples are collected from patients with or without senolytic drug treatment to assess significant changes in senescent cell phenotypes and clearance in these different tissue compartments.

The instant study is intended to characterize i) peripheral blood mononuclear cells, plasma, and serum for senescence and SASPs profiling, with and without senolytic treatment with the dietary supplement Fisetin; ii) bone marrow-derived cells and plasma for senescence and SASPs profiling, with and without Fisetin treatment; and iii) synovial cells and fluid from the knee joint for senescence and SASPs profiling, with and without Fisetin treatment. Selective elimination of senescent cells can significantly reduce the levels of SASPs, potentially enhancing musculoskeletal repair and reducing age-related disease burden.

Senescent Cells and their Senescence Associated Secretory Phenotype (SASP)

Senescent cells and their SASP are known to promote inflammation and many age-associated diseases such as diabetes, cardiovascular disease, neurodegeneration and orthopaedic related disease such as OA. Cell senescence is a fundamental mechanism by which cells are metabolically active but cease dividing and undergo distinct phenotypic changes, including upregulation of p16Ink4a (p16), significant secretome changes, telomere shortening, and decompensation of pericentromeric satellite DNA. It has been shown in naturally aged mice (24 months) that p16 expression is significantly increased in B cells, T cells, myeloid cells, osteoblast progenitor cells, osteoblasts, and osteocytes. Senotherapeutics that interfere with and delay the aging process have been demonstrated to target and modulate senescent cell and their SASP production. These include senolytics that kill senescent cells and senomorphics that modulate functions of senescent cells, inhibit (SASP) and reduce inflammation/fibrosis. The preliminary results mentioned above indicated that senescent cells that accumulate in osteoarthritic articular cartilage can be reduced after senolytic treatment.

### Senescence Markers and SASPs in Peripheral Blood, Change During the Aging Process

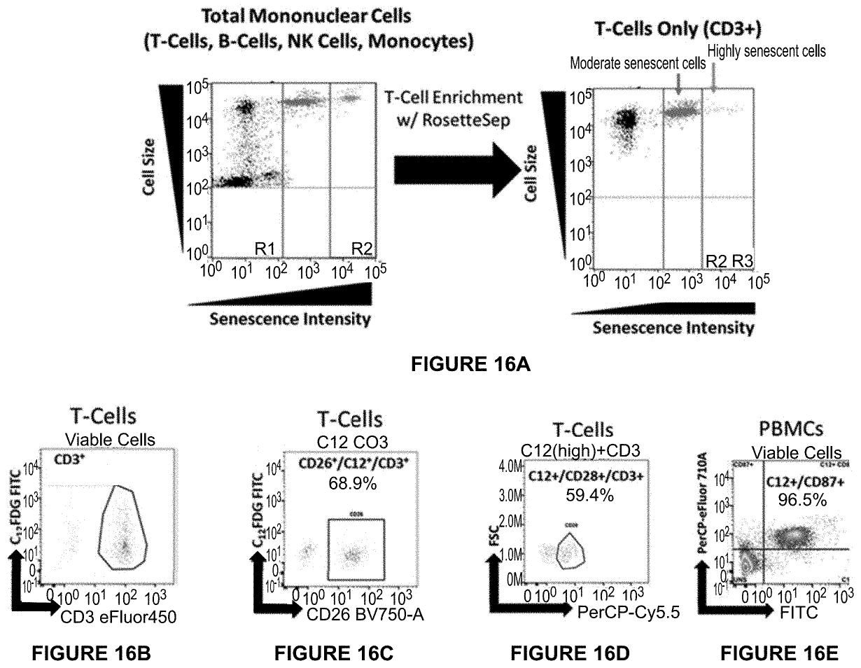

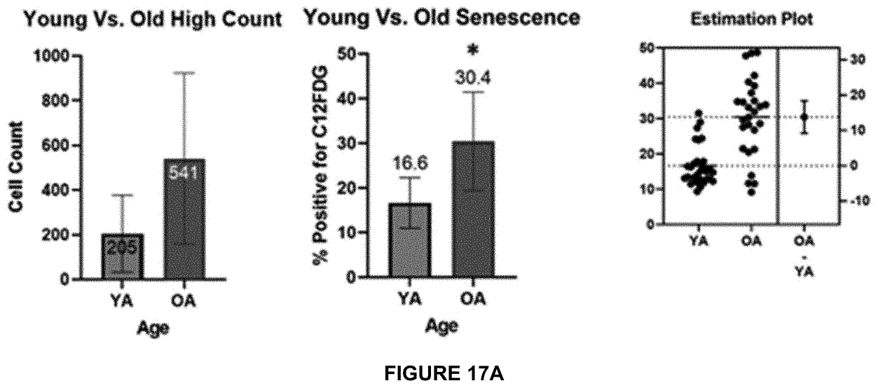

PBMCs and isolated T-Cells exhibit age-related senescence profiles. Human PBMCs, including CD3+ T-Cells, exhibit two distinct populations of senescent cells, highly senescent (high C₁₂FDG signal) and moderate or “pre-senescent” (moderate C12FDG signal) types (FIG. 16A). To support that C₁₂FDG+ senescent cells were in fact senescent, T-cells and total PBMCs were probed with antibodies targeting known senescence epitopes using spectral flow cytometry. It was found that 89.3% of the cells were in fact CD3+ supporting the negative selection technique used for T-cell purification (FIG. 16B). Furthermore, 68.9% of CD3+/C₁₂FDG+ cells co-expressed CD26 (FIG. 16C), a known senescent cell marker, while 39.2% of CD12FDG+ senescent cells exhibited loss of CD28 (FIG. 16D), a known phenotype of senescent T-cells. Total PBMCs also showed 96.5% co-localization of C₁₂FDG and CD87 (uPAR) another known senescent cell surface marker (FIG. 16E).

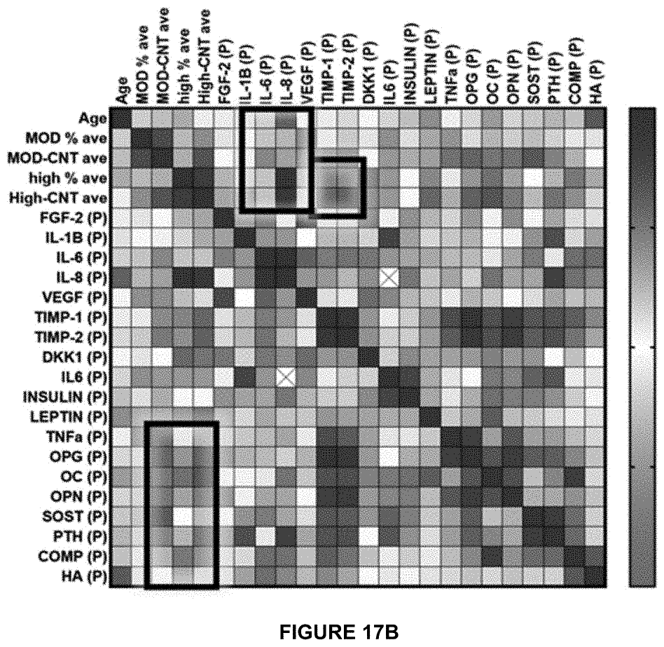

In addition, highly senescent C₁₂FDG bright PBMCs correlated with increasing chronological age of healthy donors (FIG. 17A). SASP and aging related biomarkers were also measured using multiplex immunoassays (FIG. 17B). It was found that several biomarkers were also highly co-expressed in plasma with C₁₂FDG+ cells. Several SASP factors were also detected including MCP-1 (P<0.03), IL-8 (P<0.001), VEGF (P<0.01), MMP-10 (P<0.001), TGF-β 1-2 (P<0.03), PDGF-AA (P<0.03), and TIMP-1 (P<0.03). This illustrates that this technology can be used with C₁₂FDG to identify discrete populations of PBMCs and T-cells from peripheral blood of human patients.

Senescence profiles of Bone Marrow Concentrate (BMC). Quantifiable levels of senescent cells (CD3+ T-Cells), senescence associated transcripts, and senescence associated secretory factors are assessed from samples of BMC at different ages. The results (described further below in the instant example) indicate that the number of senescent cells and SASP in BMC increased with age.

Senescence profiles of synovial fluid. Joint synovial fluid provides a source of cells and secreted factors localized to a specific compartment. Senescent cells and related SASP factors have been measured in the joint fluid of patients with knee OA (Jeon, et al. 2018 J Clin Invest 128(4):1229-37). However, specific cellular sources driving SASP and inflammation are largely unclear. The studies described herein allow for senescence and expression profiling of cells from synovial fluid to be combined with analyses of levels of SASP factors of synovium cells (synovial fibroblasts, infiltrating immune cells, and progenitor cells) and synovium derived fluid. Using C₁₂FDG staining protocol, the presence of senescent C₁₂FDG+ cells was demonstrated in synovial fluid, in patients after ACL injury with the typical 2 distinct populations of moderate C₁₂FDG signal (dim) and high C₁₂FDG signal (bright).

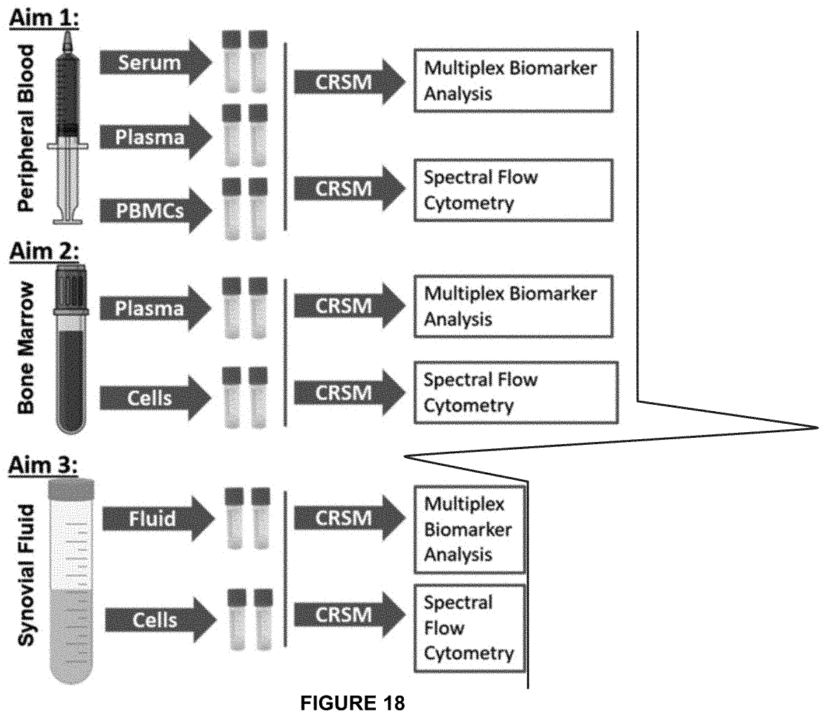

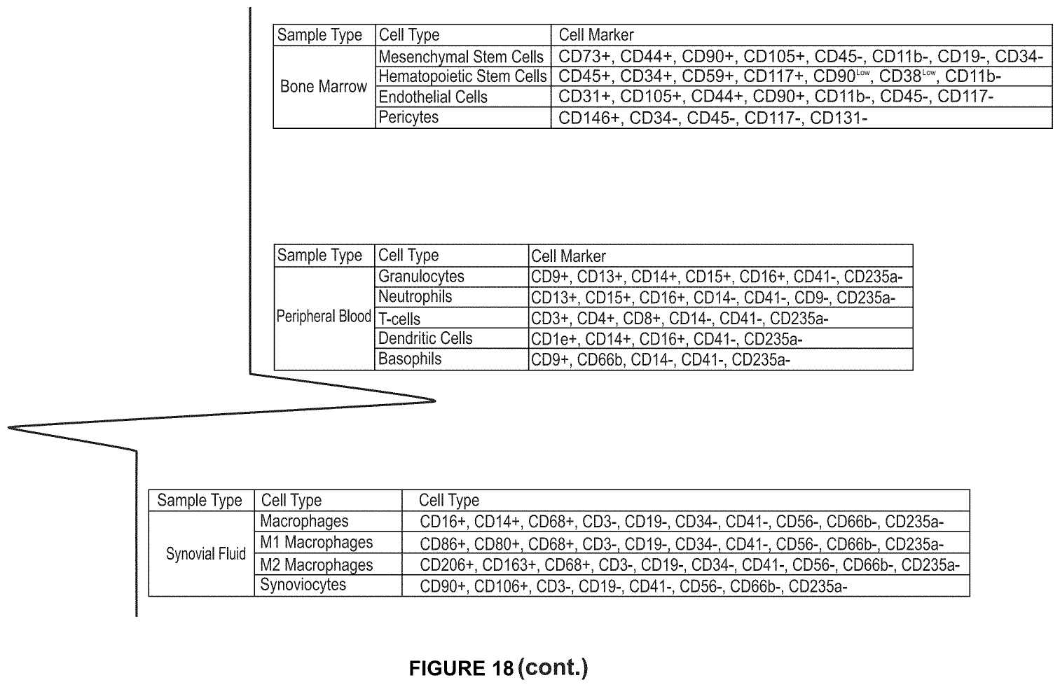

FIG. 18 provides a schematic representation of the instant study of samples (peripheral blood, bone marrow and synovial fluid) collected from the study described in Example 3, above. Tissue collection timepoints for senescence analyses are included. The acquisition, cataloging, and storage of human samples from synovial fluid, bone marrow, and peripheral blood collected from the same patient is coordinated according to established standard operating procedures (SOPs). These samples are then analyzed to perform a high-resolution molecular and functional characterization of senescent cells and their dysregulated secretome at the multi-tissue level. Senescent cell populations from these 3 different tissues compartments are isolated, identified, interrogated, and compared. SASP is assessed in acellular components using customized MAGPIX multiplex technology to detect established and novel tissue specific SASP factors. The overall design builds on the flow cytometry-based assay to detect senescent cells in human fluids, such as peripheral blood, synovial fluid and bone marrow, using the fluorescent compound C₁₂FDG (US2021/0046123A1). C₁₂FDG is a compound that, when hydrolyzed by β-galactosidase (an enzyme upregulated during senescence), fluoresces at a wavelength of 514 nm.

The senescence profile in the peripheral blood of over 180 patients has been analyzed in a clinical study (IRB 2019-58) using this flow cytometric analysis of C₁₂FDG. C₁₂FDG could be a clinically relevant biomarker that can quantify the extent of senescence within a fluid compartment based on a segregation between cells that are highly senescent (C₁₂FDG bright) versus those that are “pre-senescent” (C₁₂FDG dim). Senescent cell samples were assessed at baseline (draw 1) and after senolytic treatments (Draws 2 and 3), generating a unique data set: 189 (95 female, 94 male) participants/blood draw for Draw 1, 114 participants/blood draw for Draw 2, and 63 participants/blood draw for Draw 3. The average participant age was 53.4 years (9 were 20-30 yrs old, 13 were 30-40 yrs old, 29 were 40-50 yrs old, 36 were 50-60 yrs old, 42 were 60-70 yrs old, 46 were 70-80 yrs old, and 14 were 80-90 yrs old).

Senescent cells in the joint fluid and the bone marrow have been characterized in a number of patients, leading to the identification of two distinct populations of senescent cells (highly senescent/high C₁₂FDG signal and moderately senescent/lower C₁₂FDG signal), likely representing different stages of senescence. Thus, the described senescent cell (bright and dim) detection is not only reproducible, but also highly sensitive to detect differentiate cells at different stages of senescence in the three compartments.

5.1 Isolation and Processing of Peripheral Blood Mononuclear Cells, Plasma, and Serum for Senescence Profiling, with and without Senolytic Treatment with the Dietary Supplement Fisetin

Because senescent cell burden has been shown to strongly correlate with age-related orthopaedic conditions, and targeting and eliminating senescent cells mitigates age-related musculoskeletal decline, detecting senescent cells and their associated Senescence Associated Secretory Phenotype (SASP) factors will dramatically improve the understanding of individual patients' response to treatment and potentially assist in prescribing interventional strategies in the clinic. Thus, changes in senescent cell content and senescence biomarkers in peripheral blood mononucleated cells, plasma, and serum, with and without senolytic treatments, are analyzed.

Detection of Senescence Associated Beta Galactosidase Cells in Peripheral Blood Mononuclear Cells from Human Whole Blood Using C₁₂FDG and Flow Cytometry

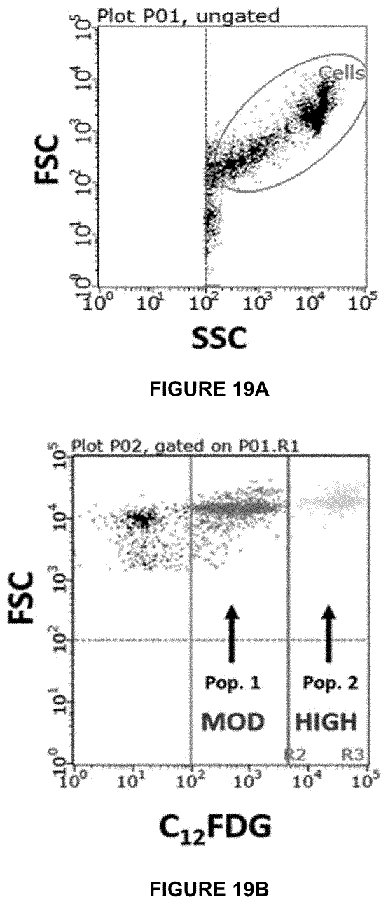

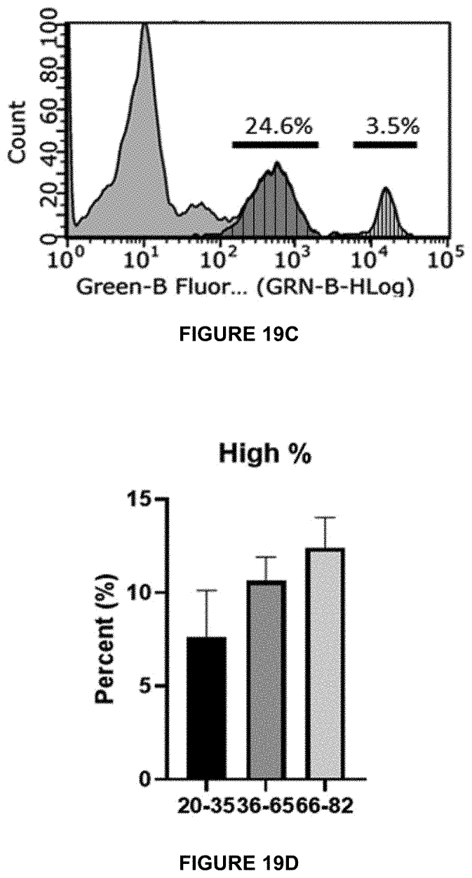

A flow cytometry-based approach to assess senescent total PBMCs from fresh human peripheral blood was optimized. Cells were identified using FSC and SSC controls (FIG. 19A), while senescent cells, or C₁₂FDG+ events, were identified with an emission of 514 nm (green channel). Of note, PBMCs displayed a distribution of two distinct populations of C₁₂FDG signal: a moderate (dim) group, potentially representing “pre-senescent” cells, and a high-brightness C₁₂FDG signal group, representing “highly senescent” cells. These highly senescent cells were found to correlate with increasing age of study participants (FIGS. 19B-19D).

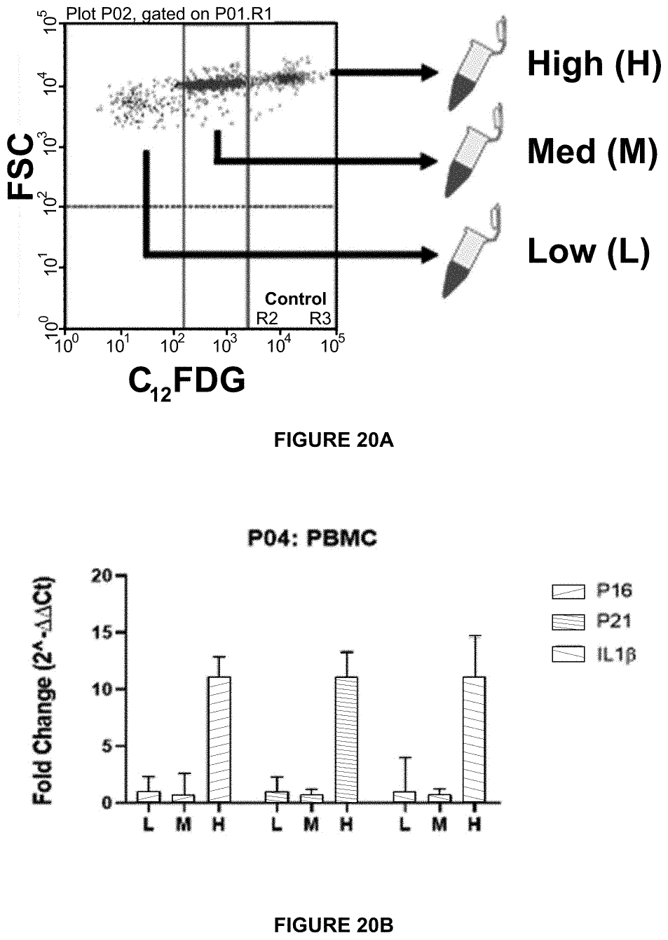

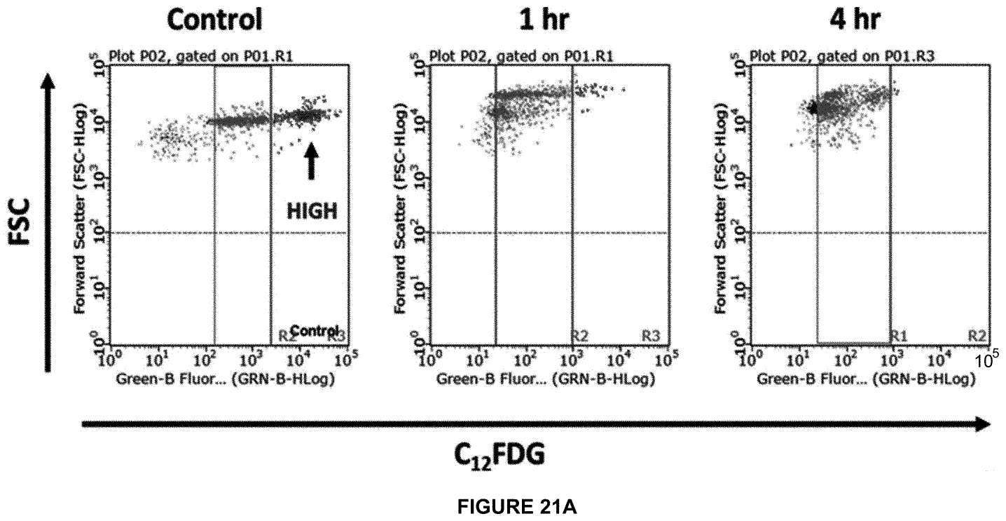

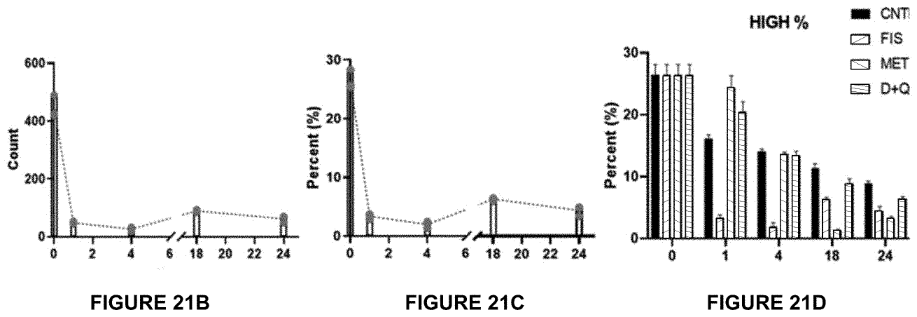

To examine cellular phenotypes of C₁₂FDG-stained cells, low, moderate, and high populations were sorted using FACS for two study participants (FIG. 20A). Indeed, expression levels for senescence/SASP markers p16INK4A and IL-1β were upregulated in highly senescent cells when compared to moderate or low senescent cells (FIG. 20B). Using the same methodology, the rate and senolytic efficacy of Fisetin treatment were determined on PBMCs directly at 1, 4, 18, and 24 hr timepoints. Surprisingly, Fisetin treatment was able to significantly reduce high senescent cell counts and percent senescent cells in as little as 1 hr, with a maximum reduction at 4 hrs (FIGS. 21A-21C). Furthermore, the rate of senolytic activity of Fisetin was faster versus other known senotherapeutic drugs such as metformin, dasatinib, or quercetin (FIG. 21D).

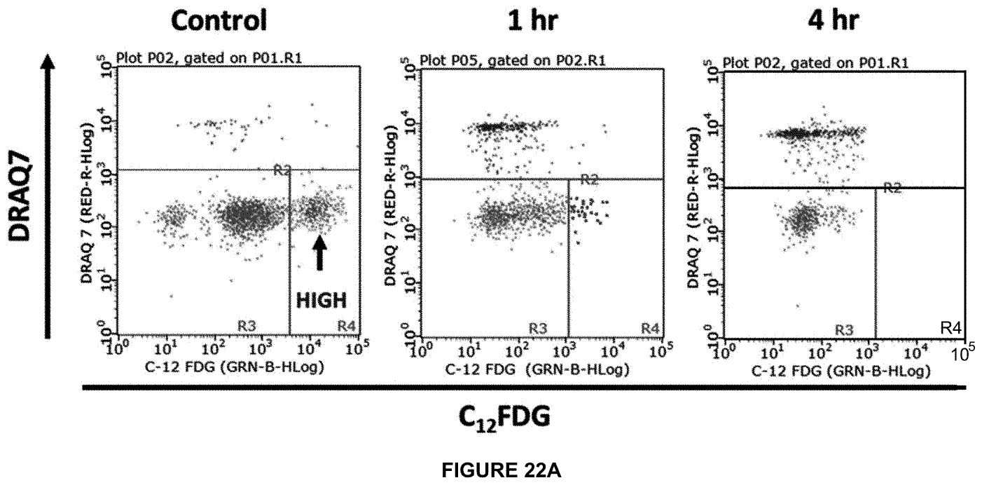

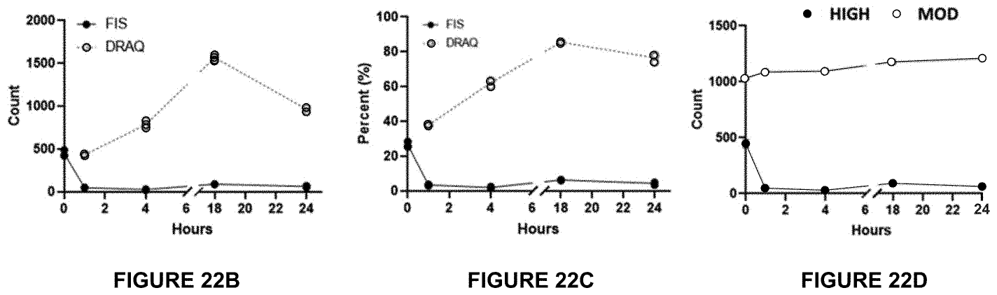

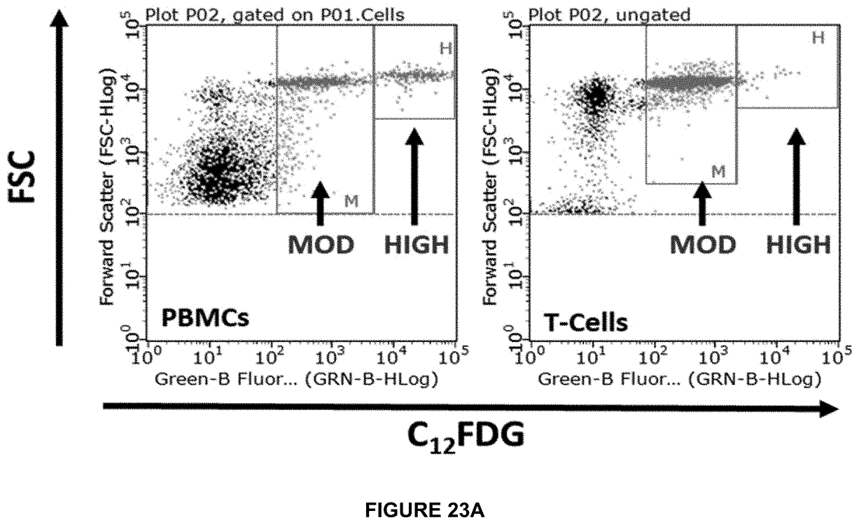

Senolytic drugs eliminate senescent cells via apoptosis through the inhibition of anti-apoptotic pathways upregulated during senescence. Thus, it was next tested whether the reduction of highly senescent cells by Fisetin was associated with co-incident cell death. To this end, cells were co-stained with the viability stain DRAQ7. Indeed, decreases in highly senescent cells (high C₁₂FDG intensity) due to Fisetin treatment were associated with concomitant increases in DRAQ7+ cells, indicating Fisetin was eliminating senescent cells through apoptosis (FIG. 22A-22C). Fisetin also seemed to primarily affect only highly senescent cells and not moderately senescent cells, suggesting minimal viability effects on healthier cells with a specificity to high senescent cell removal (FIG. 22D). Overall, these data indicate that Fisetin can rapidly eliminate senescent PBMCs from fresh human peripheral blood.

Senescent CD3+ T-Cells are Associated with Biomarkers for Age Related Orthopaedic Decline.

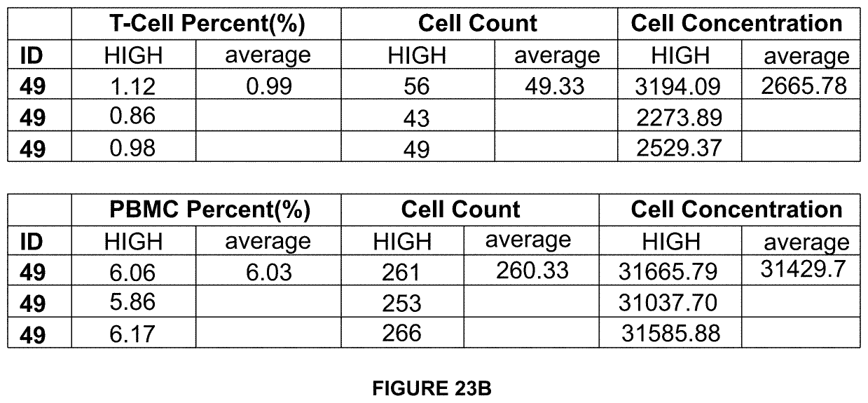

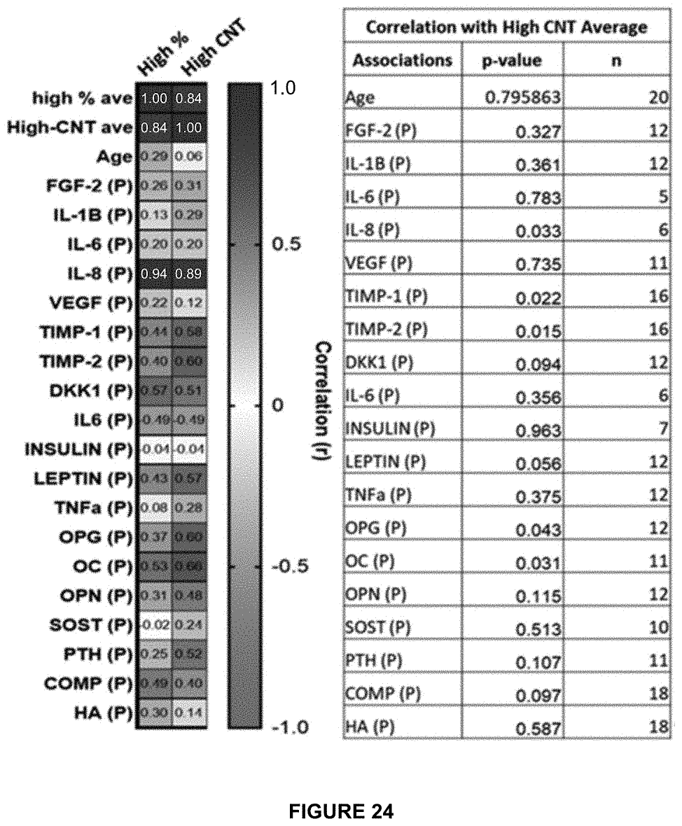

Another flow cytometry-based approach was optimized to assess senescent total PBMCs and CD3+ T-Cells from fresh human peripheral blood. For this approach, T-Cells were isolated from PBMCs, washed, then stained with the senescence marker C₁₂FDG for 1 hr, and then used for flow cytometry analysis. Cells were identified using FSC and SSC controls, while senescent cells, or C₁₂FDG+ events, were identified with an emission of 514 nm (green channel). Of note, T-Cells and PBMCs from patients displayed a distribution consisting of two distinct populations of moderate C₁₂FDG signal (moderately senescent or “pre-senescent”) and high C₁₂FDG signal (highly senescent) cell populations (FIGS. 23A and 23B). It was routinely found that highly senescent cells associated more closely with age and health status. Upon seeking to determine if highly senescent T-Cells were associated with plasma biomarkers for aging and orthopaedic health, it was found that High % and High total cell count were associated with multiple aging (SASP) markers (IL-8, TIMP1, TIMP2, Leptin) in addition to markers for osteoporosis (PTH, OPN, OPG, OC, DKK1) and osteoarthritis (HA, COMP, FGF2) (FIG. 24).

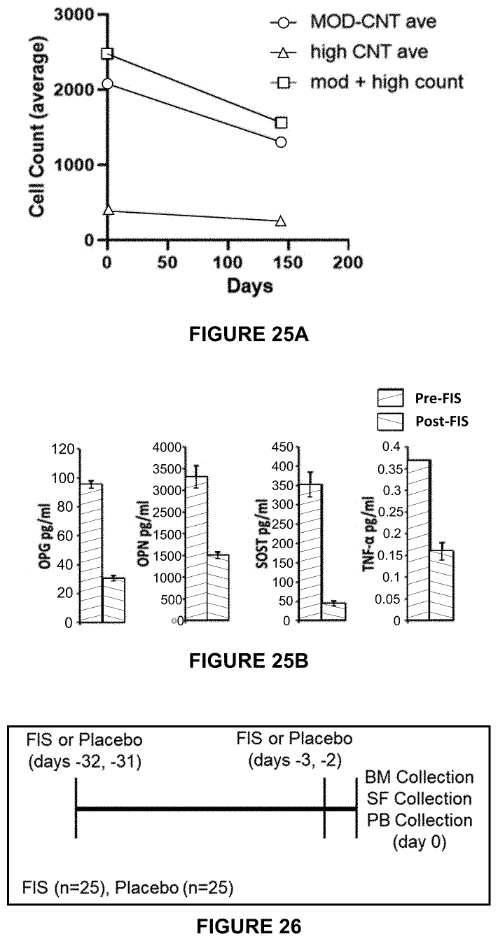

Fisetin is a naturally derived dietary supplement with demonstrated ability to eliminate senescent cells in vitro and in vivo. Thus, it was tested whether Fisetin could reduce senescent T-Cells and plasma biomarkers for OP in a single 82-year-old patient enrolled in the study that disclosed Fisetin use between blood draws. Indeed, it was found that after 150 days of Fisetin dosing (100 mg/day), levels of both moderately and highly senescent CD3+ T-Cells were reduced (FIG. 25A). This was commensurate with a reduction in OP markers OPG, OPN, and SOST in addition to the pro-inflammatory SASP marker TNF-α (FIG. 25B). Overall, these data indicate that senescent CD3+ T-Cells identified via C₁₂FDG staining may represent another biomarker for systemic aging. In addition, in a single case study, C₁₂FDG staining was sensitive enough to detect reductions in senescent T-Cells associated with reduction in OP and SASP biomarkers following Fisetin therapy.

### Experimental Design and Methodology

As mentioned above, tissue collection is carried out in conjunction the ongoing clinical trial described in Example 3. The latter so-called “parent” study includes 4 separate treatment arms (25 patients per arm) to investigate the effects of both Fisetin (a widely available dietary supplement) to reduce senescent cells and inflammation and Losartan (an FDA-approved anti-fibrotic drug) to improve the clinical efficacy of bone marrow stem cells for the treatment of knee osteoarthritis. The study described in the instant Example (5) focuses only on the two groups not treated with Losartan, in order to gain insights into the mechanistic effects of the Fisetin intervention, including the efficacy of FIS treatment for reducing senescent cells and SASP markers in bone marrow aspirate, synovial fluid, and peripheral blood. In the 2 included groups, Fisetin, or Fisetin Placebo, (oral, 20 mg/kg daily) are taken Days 32, 31, 3, and 2 prior to Bone Marrow Aspirate Injection Therapy for treating knee OA.

### Subjects

The population of the parent study includes subjects between the ages of 40 and 85 years with symptomatic knee OA (Kellgren-Lawrence grade II-IV). Exclusions for participation include clinically significant co-existing conditions that significantly compromise overall health, previous or planned surgery on the target knee, intra-articular treatment with steroids or hyaluronic acid derivatives within the last 12 weeks prior to screening, regenerative joint procedures on any joint (e.g. platelet-rich plasma injections, mesenchymal stem cell or autologous chondrocyte transplantation, mosaicplasty) within the past 6 months, current or prior history of other significant joint diseases and patients already taking a senolytic, Losartan, or closely related medications.

Peripheral Blood: The majority of senescence experiments have historically been performed in vitro, mainly in fibroblasts. However, it is becoming clearer that senescence programs can differ among different cell types and that stages of senescence exist. Senescent programs in circulating peripheral blood cells, namely PBMCs and their distinct subsets, are poorly understood and a point of debate in the field of geroscience (Xu and Larbi 2017 Int J Mol Sci. 18(8); Effros, et al. 2003 Crit Rev Immunol 23(1-2):45-64; Song, et al. 2018 Aging Cell 17(2); Vicente, et al. 2016 Aging Cell 15(3):400-6). For example, replicative senescence has been demonstrated in vitro for CD8+ T-Cells, but their ability to exhaust or senesce in vivo is unknown. Traditional epitope signatures for PBMC senescence can also be misleading and not reflective of a true non-replicative cell with increased β-Galactosidase (SA-βGal), canonical hallmarks of senescence. Numerous markers of senescence in PBMCs are characterized herein using multiple detection and analysis modalities to clearly define and map PBMC senescent states.

Sample Acquisition Pipeline: As outlined in FIG. 26, all peripheral blood (PB), bone marrow (BM), and synovial fluid (SF) samples are collected day of procedure (DOP) with or without Fisetin treatment. A total of 50 patient samples are collected (25, Fisetin; 25, placebo).

Peripheral Blood mononucleated cells (PBMC): All described blood assays are performed for a single blood draw. A standard venipuncture is performed to collect a total volume of 30 mL of blood. All tubes (red-cap serum, blue-cap plasma and cell assays) are coded with a research label including only the subjects ID # and protocol number. 0.8 mL is collected from one of the lavender tubes for hematology analysis and CBC count. The remaining volume is used for multiplex immunoassays and ELISAs (serum/plasma), Flow Cytometry, and scRNA-Seq analysis. For serum and plasma collection, blood is spun at 1,500 g for 10 minutes; then three aliquots are collected and placed in cryovials and stored at −80° C. For PBMC collection, whole blood is spun down using SepMate™ tubes using Lymphoprep™ spin medium (Stem Cell Technologies) following manufactures protocols. Isolated cells from buffy coat are assessed for viability and cell count. Table 2, below, shows established viability and concentration values to use as a reference using this methodology.

TABLE 2
Viability and concentration values of various cell types

| | Average Cell | Avg % Viablity | Avg % Viablity |
|---|---|---|---|
| Cell type | Count | FRESH (DRAQ7) | FROZEN (DRAQ7) |
| PBMC | 2.03 × 10⁶ | 94.40% | 88.50% |
| CD3+ T-Cells | 2.23 × 10⁶ | 93.80% | 87.70% |
| BMC | 1.50 × 10⁶ | 97.10% | 72.10% |
| Syn | 3.51 × 10⁵ | 98.00% | 78.20% |

Cells are then resuspended in 10% DMSO/90% FBS and slowly frozen using the ViaFreeze system (Cytivia) for downstream analysis. All samples are properly labeled with Subject ID number.

### Biomarker Analyses

A combination of high-resolution molecular and functional cellular markers (Tables 3 and 4, below) are used to assess senescence levels within the tissues under investigation, with a correlation of the tissues' acellular fluid SASP markers and the cellular component with advanced spectral flow-based cellular biomarkers.

TABLE 3
Molecular Biomarkers

| Sample | Multiplex Panel/Singleplex | |
|---|---|---|
| Type | (ELISAs) | Markers |
| Plasma, | MMP1 Panel | MMP-3, MMP-12, MMP-13 |
| Serum, | MMP2 Panel | MMP-1, MMP-2, MMP-7, MMP-9, MMP-10 |
| Synovial | TGFb-1, 2, 3 Panel | TGFb-1, TGFb-2, TGFb-3 |
| Fluid | Cytokine/Chemokine Panel | sCD40L, EGF, FGF-2, Flt-3, G-CSF, GM-CSF, GRO, IFN-α2, IFN-γ, IL-1α, IL-1β, |
| | | IL-1ra, IL-2, IL-3, IL-4, IL-5, IL-6, IL-7, IL-8, IL-9, IL-10, IL-12 (p40), IL-12 (p70), IL-13, |
| | | IL-15, IL-17A, IP-10, MCP-1, MCP-3, MDC (CCL22), MIP-1α, MIP-1β, PDGF-AB/BB, |
| | | RANTES, TGF-α, TNF-α, TNF-β, VEGF, Eotaxin/CCL11, PDGF-AA |
| | TIMP2 Panel | TIMP-1, TIMP-2, TIMP-3, TIMP-4 |
| | Aging Panel | CTACK, FGF-21, GDF-11, GDF-15, GnRH, IL-18, Jag1, Leptin |
| | Bone Panel | ACTH, DKK-1, FGF-23, Insulin, Osteocalcin, Osteopontin, Osteoprotegerin, PTH, SOST |
| | Singleplex (ELISAs) | CTX-II, HA, COMP, CS846, CRP, CHRD1, PIIANP, PAI-1, HMGB1, CGA-FSHB |

TABLE 4
Flow-Based Cellular Markers

| Sample | | |
|---|---|---|
| Type | Cell Type | Cell Marker |
| Peripheral | Granulocytes | CD9+, CD13+, CD14+, CD15+, CD16+, |
| Blood | | CD41−, CD235a− |
| | Neutrophils | CD13+, CD15+, CD16+, CD14−, CD41−, |
| | | CD9−, CD235a− |
| | T-Cells | CD3+, CD4+, CD8+, CD14−, CD41−, |
| | | CD235a− |
| | Dendritic Cells | CD1e+, CD14+, CD16+, CD41−, CD235a− |
| | Basophils | CD9+, CD66b, CD14−, CD41−, CD235a− |

A panel of +/−CD markers (Table 2) specific to each of the cell types within the tissues under investigation has been composed. Which of these cells are present in each tissue type, and which are senescent based on the use of C₁₂FDG and other flow-based markers of senescence, such as CD28 and CD87 (UPAR), are both identified. Preliminary data discussed earlier has shown C₁₂FDG to be a reliable senescence marker in the tissues. To correlate with these flow-based markers of senescence, a panel of standard phenotypic molecular biomarkers associated with senescence (Table 4, above) has been created. By correlating the SASP markers within the acellular components of each tissue with the flow-based senescent markers of the cellular components, it should be possible to identify which cell types are more likely to be senescent, and if there are correlations between the biomarkers expressed and types of senescent cells in each tissue population.

### Sample Processing and Storage

All samples are stored at −80° C. Samples are run in batches, and aliquots are stored until needed to be analyses. If statistically validated outlier analyte concentrations are found for a sample, they are re-run to try and collect useable data and remove outlier data due to human/machine procedural error. Samples are also batch processed to minimize lot differences between biomarker panels for individual patients.

### Statistical Power Analyses

An approximately 50%/50% breakdown of males and females is expected, as is a relatively uniform distribution across osteoarthritis disease grades. Additionally, a randomized 50%/50% split of subjects taking Fisetin versus those taking a placebo is produced. Between group mean differences in continuous variables will be assessed using the two-tailed Mann-Whitney U-test. Assuming a significance level of α=0.05, 25 subjects per group is sufficient to detect an effect size of Cohen's d=0.83 with 80% statistical power. Statistical analysis is performed using functions in R packages. P<0.05 will be considered statistically significant.

5.2 Isolation and Processing Bone Marrow Cells and Marrow Derived Plasma for Senescence Profiling, with and without Fisetin Treatment

Changes in senescent cells content and senescence biomarkers in bone marrow, and marrow derived plasma, with and without senolytic treatments, are analyzed.

Bone marrow contains a variety of cells including bone marrow mesenchymal stem cells, hematopoietic stem cells (HSCs), perivascular cells (PVs), and endothelial cells (ESCs). Bone marrow is also the site where hematopoiesis occurs in adults. During aging, bone marrow cells and its structure are known to change dramatically including an increase in adiposity. Alterations in gene expression are also known to occur in HSCs, consistent with senescence, as a more proinflammatory program is induced associated with functional decline and disruption of normal hematopoiesis. These changes are thought to significantly contribute to immune-senescence, a state of chronic inflammation that promotes a litany of age-related pathologies. Senescent HSCs and MSCs isolated from bone marrow of aged humans have been shown to be dysfunctional, exhibit pro-inflammatory phenotypes, and evidence of DNA damage (Gnani, et al. 2019 Aging Cell 18(3):e12933; Fali, et al. 2018 JCI Insight 3(13)). However, age-associated changes in senescent cell number and/or senescence associated markers in bone marrow have yet to be investigated. Furthermore, several studies have identified T-Cells as strong correlates to chronological age and health status (Liu, et al. 2009 Aging Cell 8(4):439-48). Thus, these cells can serve as strong cellular indicators of senescent burden in bone marrow. Finally, as mentioned previously, bone marrow aspirate concentrate (BMAC) is often used as a stem cell therapy that relies upon a natural combination of different stem cell populations residing in bone marrow with potentially unaltered niches that has been proven safe and effective for the treatment of orthopedic related symptoms. Thus, by mapping senescent cells in the bone marrow compartment, the dynamic and cells specific changes that occur with age are identified, which improves understanding and treatment strategies for BMAC-based therapies.

### Detection of Senescent Cells and SASP in Bone Marrow Aspirate

Presence of senescent cells and SASPs increase with age in bone marrow: Experiments to characterize age-related deviations in senescence markers in bone marrow aspirate (BMA) were performed from samples collected from two female osteoarthritis patients, ages 23 and 47. Following gradient centrifugation, plasma was collected and CD3+ T-Cells were enriched from PBMCs. BMA levels of several SASP factors (MMP-2,3,12 and RANTES) were found to increase significantly with age (FIGS. 2A-2D). Using flow cytometry and the fluorescent senescent cell label C₁₂FDG, the number of senescent CD3+ T-Cells was found to increase with age, even when the age difference was only 24 years (FIGS. 3A and 3B).

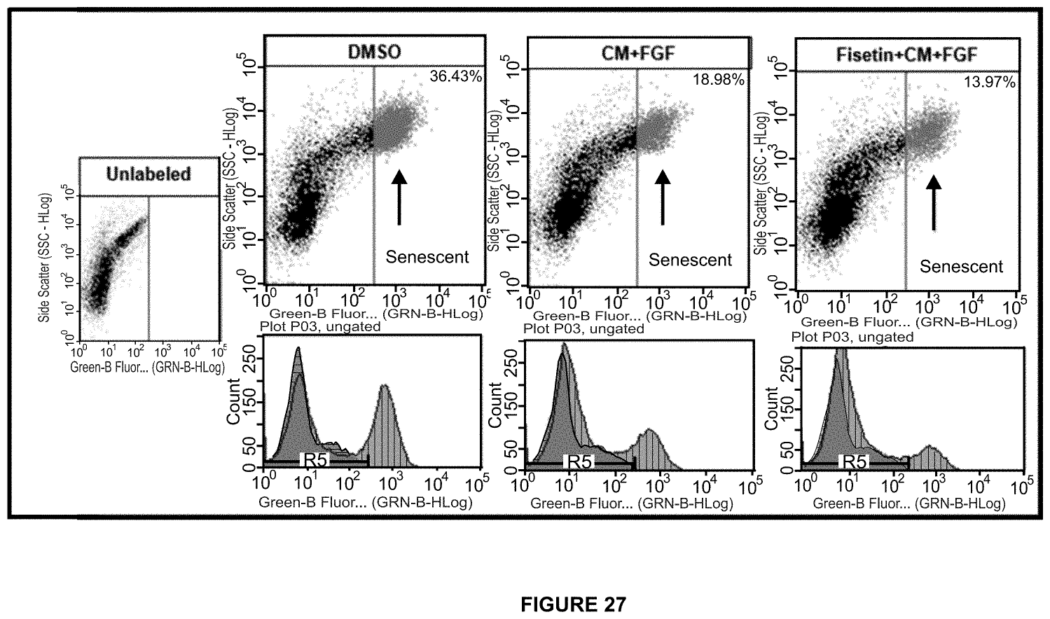

Reduction in senescent cells in BMSCs after senolytic treatment: To determine a reduction in senescent cells in bone marrow stem cells after senolytic therapy, approximately 5 mL of bone marrow concentrate were obtained from a 57-year-old male. The BMC samples were immediately processed further to isolate the mononuclear layer and plated in a T25 flask to expand BM-MSCs. Bone marrow cells were pre-plated for 3-weeks in basal culture medium (CM) and passaged with CM+Fibroblast Growth Factor (FGF) to expand bone marrow mesenchymal stem cells (BM-MSCs). At passage 3, confluent BM-MSCs were treated with CM+FGF, 33 μM of DMSO, or 33 μM of Fisetin+GM+FGF for 24-hours in a 12-well plate. Then, 50 mM of bafilomycin (inhibits lysosomal acidification) and 3304 of C₁₂FDG (senescent label) were used to assess senescence via flow cytometry. In BM-MSCs at passage 3, the number of senescent in the Fisetin treated BM-MSCs was reduced by approximately 24% compared to the DMSO treated BM-MSCs, and approximately a 5% reduction of senescent cells was observed when compared to control BM-MSCs (FIG. 27). These results taken together show that senescent cells and SASP can be measured within bone marrow cells, and that senescent cells can be eliminated with senolytic treatment (fisetin).

### Experimental Design and Methodology

Tissue and subject selection, IRB (inclusion and exclusion criteria), scientific justification for tissues, sample acquisition pipeline, processing and storage, and biomarkers analyses are performed in a similar manner as described in Example 5.1.

Bone Marrow isolation and characterization: A minimal volume of 1 ml (maximum of 2 ml) bone marrow aspirate concentrate is collected on the day of procedure (DOP). For bone marrow harvest, BMA is aspirated from the posterior-superior iliac crest, as previously described (Chahla, et al. 2017 Arthrosc Tech 6(2):e441-e5). A volume of 90-120 mL of BMA is harvested per standard-of-care from either or both the left and right sides of the posterior-superior iliac crest. The BMA is centrifuged using a benchtop centrifuge at 1,500 g for 10 minutes. The cellular pellet and acellular fluid are preserved. The BMA is then filtered through an 18-micron filter into a conical tube to filter potential clots. A second centrifugation is performed at 3,000 g for 6 minutes. BMC is platelet depleted (PluriSpin), then spun to collect buffy coat cells using sponge column tubes (PluriSelect). The top layer of plasma (BMC-P) is collected in three aliquots (˜1 mL total) for protein assays. Isolated bone marrow cells are assessed for viability and cell count. The viability and concentration values of Table 2, above, are used as a reference. Isolated cells are characterized for the presence of mesenchymal stem cells, hematopoietic stem cells, endothelial cells and pericytes, using specific markers (see Table 5, below) and are resuspended in 10% DMSO/90% FBS, then slowly frozen using ViaFreeze system (Cytivia) for downstream analysis.

TABLE 5
Flow-Based Cellular Markers

| Sample | | |
|---|---|---|
| Type | Cell Type | Cell Marker |
| Bone | Mesenchymal | CD73+, CD44+, CD90+, CD105+, CD45−, |
| Marrow | Stem Cells | CD11b−, CD19−, CD34− |
| | Hematopoietic | CD45+, CD34+, CD59+, CD117+, |
| | Stem Cells | CD90^{Low}, CD38^{low}, CD11b−, |
| | Endothelial | CD31+, CD105+, CD44+, CD90+, CD11b−, |
| | Cells | CD45−, CD117− |
| | Pericytes | CD146+, CD34−, CD45−, CD117−, CD31− |

5.3 Isolation and Processing Synovial Cells and Fluid from the Knee Joint for Senescence Profiling, with and without Fisetin Treatment

Changes in senescent cells content and senescence biomarkers in synovial cells and fluid, with and without senolytic treatments, are analyzed.

Joint synovial fluid provides a source of cells and secreted factors localized to a specific compartment. Senescent cells and related SASP factors have been measured in the joint fluid of patients with knee OA (Jeon, et al. 2018 J Clin Invest 128(4):1229-37). However, specific cellular sources driving SASP and inflammation are largely unclear. The studies described herein allow for senescence and expression profiling with concentration levels of SASP factors to clarify specific secretome profiles for senescent synovium cells such as synovial fibroblasts, infiltrating immune cells, and progenitor cells.

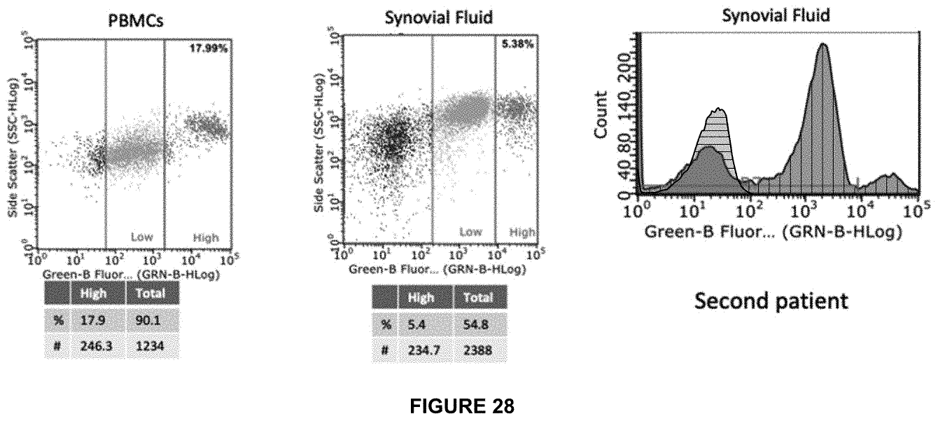

Senescence Detection in Synovial Fluid: 4-30 mL of synovial fluid was transferred in anticoagulant and centrifuged at 1,500 g for 10 minutes. Cells were identified using FSC and SSC controls, while senescent cells, or C₁₂FDG+ events, were identified with an emission of 514 nm (green channel). Of note, synovial fluid senescent cells labelling also displayed a distribution of two distinct populations of moderate C₁₂FDG signal (Dimmed) potentially “pre-senescent cells”, and high C₁₂FDG signal or “highly senescent cells” (FIG. 28).

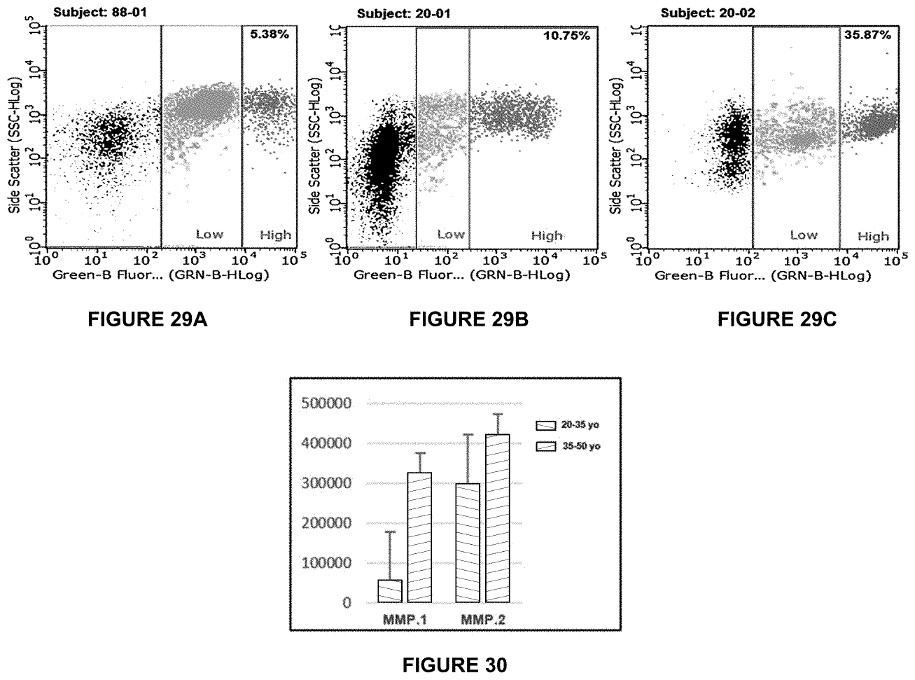

Variation of Senescence Detection in Synovial Fluid between subject and after Injury: Data in FIGS. 29A-29C represents detection of senescent cells using C₁₂FDG and Draq7 in synovial fluid from 2 separate patients (88 and 20) that had sustained an acute knee injury within 48 hours to 6 weeks. Subject 88 underwent a single knee aspiration procedure within 48 hours of injury (88-01). Subject 20 underwent an aspiration procedure within 48 hours (20-01) and at the time of surgery within 6 weeks from injury (20-02). A difference in the number of senescent cells has been observed between the subjects (FIGS. 29A and 29B). An increase in senescent cells was observed between the 1st aspiration and 2nd aspiration procedures (performed 12 days apart) in subject 20 (FIGS. 29B and 29C).

SASP associated biomarkers within synovial fluid samples: SASP associated biomarkers were also analyzed within synovial fluid samples from acute knee injured patients between 20-50 years of age. Synovial fluid samples were collected one time point (intra-operatively) from patients with acute anterior cruciate ligament injury (from <1 week of injury). Synovial fluid samples were assayed and analyzed using the Luminex 200® multiplex instrument. FIG. 30 shows that SASP (MMP1 and MMP2) can be detected in joint fluid and older patients contain more SASP factors than younger patients. These results taken together show that senescent cells and SASP can be detected in joint fluid and that variation between subject and within the same subject at different time after injury can be measured.

### Experimental Design and Methodology

Tissue and subject selection, IRB (inclusion and exclusion criteria), scientific justification for tissues, sample acquisition pipeline, processing and storage, and biomarkers analyses are performed in a similar manner as described in Example 5.1.

Joint fluid isolation and characterization: A minimal volume of 5 mL (maximum of 7 ml) is collected via arthrocentesis from the study knee on the day of procedure (bone marrow aspiration). Once the skin is anesthetized, synovial fluid is collected into sterile syringes using a lateral suprapatellar approach with a needle, and then immediately transferred to a sterile centrifuge tube under a biosafety hood. 4-30 mL of synovial fluid was transferred in anticoagulant for further processing. Under a hood, 3-4 mL of synovial fluid is transferred to a 15 mL conical tube for collection of acellular synovial fluid for biomarker analysis. The remaining sample is distributed to a separate 15 or 50 mL conical tube for senescence staining procedures. In the senescent staining conical tube, the sample is diluted with an equal volume of PBS with 2% FBS. The conical tubes are centrifuged at 1,500 g for 10 minutes. From the biomarker 15 mL conical tube, 50 mL to 2000 mL of the acellular portion of synovial fluid are collected and transferred to microcentrifuge tubes and store at −80° C. for biomarker analysis. The remaining acellular layer is discarded. The cell pellet is resuspended in 1 of 4, 5 mL conical tubes in 1 mL of cryopreservation solution (StemCell Technologies, Vancouver, BC) and transferred to a cryovial. In the 2nd 5 mL conical tube is resuspended the cell pellet in 1 mL of tryzol and transferred to a cryovial. 2/4 cryovials are stored at −80° C. until batch analysis. The remaining 2/4 cell pellets will be resuspended in DMEM/F12 with 10% FBS and 1% pen/strep and performed cell count using trypan blue stain. Then, 10 μM of bafilomycin (Sigma-Aldrich, St. Louis, Mo.) and/or 3 μM of Draq7® (Biostatus Ltd, Shepshed, UK) are added to both 5 mL conical tubes and incubated at 37° on a shaker for 1 hour. After 1 hour, 3304 of C₁₂FDG are added to one of the 5 mL conical tubes (labeled) and incubated at 37° C. for 1 hour. Cells in the unlabeled (control) and labeled conical tubes are washed with PBS two times and centrifuged at 800 g for 5 minutes. Cell pellets are resuspended in PBS and individually loaded into each well (100 μL sample/100 μL of PBS) on a 96-well plate for immediate analysis (Guava®, EasyCyte, Hayward, Calif.). Residual sample from the unlabeled tube will be centrifuged at 800 g for 5 minutes and resuspended in cryopreservation solution (StemCell Technologies, Vancouver, BC) and stored at −80° C. for future analysis. Isolated synovial cells from pellet are assessed for viability and cell count/characterization. The viability and concentration values of Table 2, above, are used as a reference. Cells from buffy coat are characterized as described in Table 6, below, and resuspended in 10% DMSO/90% FBS, then slowly frozen using ViaFreeze system (Cytivia) for downstream analysis. All samples are properly labeled with a Subject ID number.

TABLE 6
Flow-Based Cellular Markers

| Sample | | |
|---|---|---|
| Type | Cell Type | Cell Marker |
| Synovial | Macrophages | CD16+, CD14+, CD68+, CD3−, CD19−, |
| Fluid | | CD34−, CD41−, CD56−, CD66b−, CD235a− |
| | M1 | CD86+, CD80+, CD68+, CD3−, CD19−, |
| | Macrophages | CD34−, CD41−, CD56−, CD66b−, CD235a− |
| | M2 | CD206+, CD163+, CD68+, CD3−, CD19−, |
| | Macrophages | CD34−, CD41−, CD56−, CD66b−, CD235a− |
| | Synoviocytes | CD90+, CD106+, CD3−, CD19−, CD41−, |
| | | CD56−, CD66b−, CD235a− |

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method for enhancing a therapeutic outcome in a subject having a musculoskeletal condition or disorder, comprising administering at least one senolytic agent and/or at least one anti-fibrotic agent to the subject.

2. The method of claim 1, wherein the therapeutic outcome is related to the outcome of surgical and/or non-surgical treatment of the musculoskeletal condition or disorder.

3. The method of claim 1, wherein the musculoskeletal condition or disorder is a bone injury, a bone condition, or a bone disorder.

4. The method of claim 2, wherein the non-surgical treatment comprises administration of an orthobiologic to the subject.

5. The method of claim 4, wherein the non-surgical treatment comprises administration of bone marrow stem cells to the subject.

6. The method of claim 5, wherein the musculoskeletal condition or disorder is osteoarthritis.

7. The method of any one of claims 1-6, wherein the at least one senolytic agent is Fisetin.

8. The method of any one of claims 1-6, wherein the at least one anti-fibrotic agent is Losartan.

9. The method of any one of claims 1-6, wherein the at least one senolytic agent is Fisetin, and the at least one anti-fibrotic agent is Losartan.

10. The method of claim 7 or 9, wherein the senolytic agent is administered to the subject in cycles of about 2 days on/about 28 days off before treatment.

11. The method of claim 7 or 9, wherein the senolytic agent is administered to the subject in cycles of about 2 days on/about 28 days off before and after treatment.

12. The method of claim 8 or 9, wherein the anti-fibrotic agent is administered for at least about 30 days after treatment.

13. The method of claim 9, wherein the senolytic agent is administered to the subject in cycles of about 2 days on/about 28 days off before treatment, and the anti-fibrotic agent is administered for at least about 30 days after treatment.

14. The method of claim 9, wherein the senolytic agent is administered to the subject in cycles of about 2 days on/about 28 days off before and after treatment, and the anti-fibrotic agent is administered for at least about 30 days after treatment.

15. The method of claim 7 or 9, wherein the at least one senolytic agent is administered at a dosage of about 1000 mg/day.

16. The method of claim 7 or 9, wherein the at least one senolytic agent is administered at a dosage of about 10 mg/kg/day to about 100 mg/kg/day.

17. The method of claim 16, wherein the at least one senolytic agent is administered at a dosage of about 20 mg/kg/day.

18. The method of claim 8 or 9, wherein the at least one anti-fibrotic agent is administered at a dosage of about 10 mg/day to about 200 mg/day.

19. The method of claim 18, wherein the at least one anti-fibrotic agent is administered at a dosage of about 25 mg/day.

20. A method for improving the outcome of bone marrow stem cell (BMSC) treatment of symptomatic knee osteoarthritis in a subject, comprising combining the BMSC treatment with administration of at least one senolytic agent and/or at least one anti-fibrotic agent to the subject.

21. The method of claim 20, wherein the at least one senolytic agent is Fisetin.

22. The method of claim 20, wherein the at least one anti-fibrotic agent is Losartan.

23. The method of claim 20, wherein the at least one senolytic agent is Fisetin, and the at least one anti-fibrotic agent is Losartan.

24. A method for reducing the senescent cell content of the peripheral blood mononucleated cells, plasma, and/or serum of a subject having symptomatic knee osteoarthritis, comprising administering a senolytic agent to the subject.

25. A method for reducing the senescent cell content of the bone marrow and/or marrow-derived plasma of a subject having symptomatic knee osteoarthritis, comprising administering a senolytic agent to the subject.

26. A method for reducing the senescent cell content of the synovial cells and/or synovial fluid of a subject having symptomatic knee osteoarthritis, comprising administering a senolytic agent to the subject.

27. The method of any one of claims 1-26, further comprising measuring senescent cells in a sample obtained from the subject before and/or after treatment.

28. The method of claim 27, wherein the sample is selected from the group consisting of peripheral blood mononucleated cells, plasma, serum, bone marrow, marrow-derived plasma, synovial cells, and synovial fluid.

29. A kit for use in enhancing a therapeutic outcome in a subject having a musculoskeletal condition or disorder, the kit comprising at least one senolytic agent and/or at least one anti-fibrotic agent.

30. The kit of claim 29, wherein the therapeutic outcome is related to the outcome of surgical and/or non-surgical treatment of the musculoskeletal condition or disorder.

31. The kit of claim 29 or 30, wherein the at least one senolytic agent is Fisetin.

32. The kit of claim 29 or 30, wherein the at least one anti-fibrotic agent is Losartan.

33. The kit of claim 29 or 30, wherein the at least one senolytic agent is Fisetin, and the at least one anti-fibrotic agent is Losartan.
